(19) United States
(12) Patent Application Publication
(10) Pub. No.: US 2023/0312610 A1
(43) Pub. Date: Oct. 5, 2023

(54) Anti-Microbial Compositions

(71) Applicant: 5D HEALTH PROTECTION GROUP LTD, Gwynedd (GB)

(72) Inventors: Rui Chen, Liverpool (GB); Steve Law, Liverpool (GB); Steven Percival, Liverpool (GB)

(21) Appl. No.: 18/006,744

(22) PCT Filed: Jul. 27, 2021
(86) PCT No.: PCT/GB2021/051922

§ 371 Date: Jan. 25, 2023

(30) Foreign Application Priority Data
Jul. 27, 2020 (GB) 2011581.2

(51) Int. Cl. A01N 37/44; A01N 59/06; A01N 59/16; C07F 1/10

(52) U.S. Cl. CPC C07F 1/10; A01N 37/44; A01N 59/06; A01N 59/16

(57) ABSTRACT

The present invention relates to compounds, compositions, methods of forming/preparing such compounds and compositions, and uses for sanitising and/or substantially removing biofilms and microorganisms living within or around biofilms. The present invention, in particular, relates to compounds of formula Mn(P) as described herein which can be used in wound treatments, wound dressings, medical devices, water treatments, food processing and dental care biofilms.

### FIELD OF THE INVENTION

The present invention relates to compounds, compositions, methods of forming/preparing such compounds and compositions, and uses for sanitising and/or substantially removing biofilms and microorganisms living within or around biofilms. The present invention, in particular, relates to compounds of formula Mₙ(P) as described herein which can be used in wound treatments, wound dressings, medical devices, water treatments, food processing and dental care biofilms.

### BACKGROUND OF THE INVENTION

The colonisation of surfaces (abiotic and biotic), including skin and medical devices, by microorganisms represents a significant infection risk to a patient. When these microorganisms attach and grow on a surface (abiotic and biotic) they form a biofilm. It is increasingly recognised that microbial populations living within a biofilm environment contribute to delayed healing and increased infection risk. One strategy used to prevent or reduce the formation of biofilms on a surface is to remove vital materials required by bacteria such as various metal ions like iron and zinc. It has been established that by exposing the biofilm to compounds that preferentially bind to the metal ions, or even extract them from growing bacterial colonies, the biofilm growth can be curtailed. One class of such compounds are metal chelates such as polyacids, like citric acid, polyphosphates and polyaminocarboxylates. Materials formed from ethylenediaminetetraacetic acid (EDTA) and a mixture of two or more metal ions to provide mixed metal EDTA complexes, have been described (WO2017/191453) and demonstrate powerful activity against biofilm.

### SUMMARY OF THE INVENTION

Whilst the EDTA compounds described in WO2017/191453 have been observed to be effective anti-microbial and anti-biofilm compounds, and can be utilised in a wide range of products, the present invention proposes an alternative class of mixed metal aminopolycarboxyl complexes that show an unexpected improvement in anti-microbial and anti-biofilm efficacy amongst other beneficial properties.

The present inventors have discovered that by using certain aminopolycarboxyl components, for example diaminotetraethylenepentaacetic acid (DTPA) or tetraethylenediaminehexaacetic acid (TTHA), together with two or more metal ions, as defined herein, it is possible to provide mixed metal complexes which possess unexpectedly superior properties as compared to the known mixed metal EDTA complexes previously disclosed in WO2017/191453. Specifically, the efficacy of the mixed metal aminopolycarboxyl complexes of the present invention is significantly better than the known mixed metal EDTA complexes. This allows for a lower loading of the mixed metal aminopolycarboxyl complexes of the present invention into target products (e.g. wound dressings, medicaments, etc.) leading to a variety of benefits. One such benefit includes increased cost saving benefits in the manufacture of target products containing the mixed metal aminopolycarboxyl complexes of the present invention. This also helps overcome the manufacturing and scale up challenges associated with the comparatively poorly soluble EDTA mixed metal complexes described in WO2017/191453.

Importantly, the improvement in the mixed metal aminopolycarboxyl complexes of the present invention is not predictable when measuring the efficacy of the uncomplexed equivalents of the aminopolycarboxyl components utilised in the present invention as compared to uncomplexed EDTA equivalents. This is discussed in more detail herein.

Accordingly, in a first aspect, the invention provides compound of formula Mn(P), wherein:
n is an integer from 2 to 6;
each M is independently a metal ion;
Mn comprises at least two different metal ions selected from Ag, Al, Au, Ba, Bi, Tl, Ce, Co, Cu, Fe, Ga, Ge, Ir, Mo, Rh, Ru, Sb, Se, Sn, Sr, Ti and Zn ions; and P is an aminopolycarboxyl component comprising an optionally substituted alkylene amino backbone containing from 3 to 5 nitrogen atoms in the backbone and 5 or 6 carboxyl groups appended to the backbone, wherein the aminopolycarboxyl component contains from 10 to 20 atoms in the longest linear chain.

In a second aspect, the invention provides a composition comprising one or more compounds according to the first aspect of the present invention and any embodiment thereof described herein, and optionally further including one or more pharmaceutically acceptable excipients.

In a third aspect, the invention provides a method of forming a compound of formula Mn(P), wherein:
n is an integer from 2 to 6;
each M is independently a metal ion;
Mn comprises at least two different metal ions selected from Ag, Al, Au, Ba, Bi, Tl, Ce, Co, Cu, Fe, Ga, Ge, Ir, Mo, Rh, Ru, Sb, Se, Sn, Sr, Ti and Zn ions; and P is an aminopolycarboxyl component comprising an optionally substituted alkylene amino backbone containing from 3 to 5 nitrogen atoms in the backbone and 5 or 6 carboxyl groups appended to the backbone, wherein the aminopolycarboxyl component contains from 10 to 20 atoms in the longest linear chain
the method comprising:
providing a solution comprising P;
contacting the solution comprising P with a first metal ion source comprising a first metal ion M and a second metal ion source comprising a second metal ion M to form the compound of formula Mn(P);
wherein the first metal ion M and second metal ion M are independently selected from Ag, Al, Au, Ba, Bi, Tl, Ce, Co, Cu, Fe, Ga, Ge, Ir, Mo, Rh, Ru, Sb, Se, Sn, Sr, Ti and Zn ions, and the first metal ion M is different to the second metal ion M.

In a fourth aspect, the invention provides a method of preparing an anti-microbial composition comprising at least two different metal ions selected from Ag, Al, Au, Ba, Bi, Tl, Ce, Co, Cu, Fe, Ga, Ge, Ir, Mo, Rh, Ru, Sb, Se, Sn, Sr, Ti and Zn ions and a component P, wherein P is an aminopolycarboxyl component comprising an optionally substituted alkylene amino backbone containing from 3 to 5 nitrogen atoms in the backbone and 5 or 6 carboxyl groups appended to the backbone, wherein the aminopolycarboxyl component contains from 10 to 20 atoms in the longest linear chain, the method comprising:
providing a solution comprising component P;
contacting the solution comprising component P with a first metal ion source comprising a first metal ion M selected from Ag, Al, Au, Ba, Bi, Tl, Ce, Co, Cu, Fe, Ga, Ir, Mo, Rh, Ru, Ti and Zn ions and a second metal ion source comprising a second metal ion M that is different to the first metal ion M, the second metal ion M selected from Ag, Al, Au, Ba, Bi, Tl, Ce, Co, Cu, Fe, Ga, Ge, Ir, Mo, Rh, Ru, Sb, Se, Sn, Sr, Ti and Zn ions, to form the anti-microbial composition;
wherein the molar ratio of the component P, first metal ion source and second metal ion source (P : first metal ion : second metal ion) is from about 1 : 2 : 2 to about 1 : 10 : 10 or from about 1 : 0.1 : 0.1 to about 1 : 10 : 10.

In a fifth aspect of the present invention there is a provided an anti-microbial composition obtainable by the method according to the fourth aspect of the present invention and any embodiment thereof described herein.

In a sixth aspect, the invention provides a wound dressing or medical device comprising a compound according to the first aspect of the present invention or any embodiment thereof, a composition according to the second aspect of the present invention or any embodiment thereof or a composition according to the fifth aspect of the present invention or any embodiment thereof as described herein.

In a seventh aspect, the present invention provides use of the compound according to the first aspect of the present invention or any embodiment thereof, the composition according to the second aspect of the present invention or any embodiment thereof or a composition according to the fifth aspect of the present invention or any embodiment thereof as described herein to sanitise and/or substantially remove a biofilm from a substrate wherein the use excludes using the compound or composition in a method for treatment of the human or animal body by surgery or therapy. For instance, it may be that the substrate is not a human or animal body, or component thereof.

In an eighth aspect, the present invention provides a compound according to the first aspect of the present invention or any embodiment thereof, a composition according to the second aspect of the present invention or any embodiment thereof or a composition according to the fifth aspect of the present invention or any embodiment thereof as described herein for use as a medicament.

In a ninth aspect, the present invention provides a compound according to the first aspect of the present invention or any embodiment thereof, a composition according to the second aspect of the present invention or any embodiment thereof or a composition according to the fifth aspect of the present invention or any embodiment thereof as described herein for use in a method of sanitising and/or substantially removing a biofilm from a substrate, optionally wherein the substrate is a wound on a human or animal body.

A tenth aspect of the present invention provides a compound according to the first aspect of the present invention or any embodiment thereof, a composition according to the second aspect of the present invention or any embodiment thereof or a composition according to the fifth aspect of the present invention or any embodiment thereof as described herein for use in a method of treating infections of cuts, bruises, surgical sites, lacerations, abrasions, punctures, incisions, gunshots, burns, pyoderma, atopic dermatitis, eczema, pressure ulcers, venous and artery leg ulcers, diabetic foot ulcers, cystic fibrosis (CF)-associated infections, mastitis, otitis, community or hospital acquired infections or food-borne diseases.

### DETAILED DESCRIPTION OF THE INVENTION

### Compounds of the Present Invention

Compounds of the formula Mₙ(P) as disclosed herein may be as described with reference to the definitions provided below.

Compounds of formula Mₙ(P) as described herein are examples of metal aminopolycarboxyl complexes or compounds. The term “metal aminopolycarboxyl compound” refers to any type of chemical species which comprises at least one metal and at least one aminopolycarboxyl component or molecule, where said metal and said aminopolycarboxyl component are present as salts, ions or neutral species and therefore encompasses metal aminopolycarboxyl salts, chelates and co-ordination complexes. The compound of formula Mₙ(P) may be a salt and/or a chelate and/or a co-ordination complex. Suitably the compound of formula Mₙ(P) is a salt comprising ionic bonds between the metal and the aminopolycarboxyl components. Suitably, the compound of formula Mₙ(P) is a neutral compound. It may be assumed that, where no charge information is given in a particular formula, the compound represented by that formula has no overall charge.

The compound may be in the form of a solid. Alternatively, the compound may be present in a solution or a suspension, suitably an aqueous solution or an aqueous suspension. In embodiments, the compound may be present in an aqueous solution.

### Mₙ Group

Compounds according to the invention contain a plurality “n” of metal ions “M”. In accordance with any of the aspects or embodiments described herein n is an integer from 2 to 6. In embodiments, n may be 2, 3, 4, 5 or 6. Preferably, n is 2, 3 or 4. n may be 2. n may be 3. n may be 4. The compounds of formula Mₙ(P) according to the present invention may therefore be di-metal aminopolycarboxyl compounds, tri-metal aminopolycarboxyl compounds, tetra-metal aminopolycarboxyl compounds, penta-metal aminopolycarboxyl compounds or hexa-metal aminopolycarboxyl compounds.

In accordance with any of the aspects or embodiments referred to herein, each M may independently be any metal ion suitable for forming compounds of Mₙ(P) of the present invention, provided that Mₙ comprises at least two different metal ions selected from Ag, Al, Au, Ba, Bi, Tl, Ce, Co, Cu, Fe, Ga, Ge, Ir, Mo, Rh, Ru, Sb, Se, Sn, Sr, Ti and Zn ions. In other words, Mₙ comprises at least a first metal ion based on a first element selected from Ag, Al, Au, Ba, Bi, Tl, Ce, Co, Cu, Fe, Ga, Ge, Ir, Mo, Rh, Ru, Sb, Se, Sn, Sr, Ti and Zn and a second metal ion based on a second element selected from Ag, Al, Au, Ba, Bi, Tl, Ce, Co, Cu, Fe, Ga, Ge, Ir, Mo, Rh, Ru, Sb, Se, Sn, Sr, Ti and Zn wherein the first and second metals are different. For the avoidance of doubt, a compound of the formula Mₙ(P) may be considered to comprise at least a first metal ion M¹ and at least a second metal ion M², wherein M¹ and M² are different and each are independently selected from Ag, Al, Au, Ba, Bi, Tl, Ce, Co, Cu, Fe, Ga, Ge, Ir, Mo, Rh, Ru, Sb, Se, Sn, Sr, Ti and Zn ions. In some embodiments of the present invention, Mₙ may comprise at least two different metal ions independently selected from Ag, Al, Bi, Cu and Zn ions.

In this regard, in addition to the at least two different metal ions selected from Ag, Al, Au, Ba, Bi, Tl, Ce, Co, Cu, Fe, Ga, Ge, Ir, Mo, Rh, Ru, Sb, Se, Sn, Sr, Ti and Zn ions, Mn may comprise other metals ions which are not selected from Ag, Al, Au, Ba, Bi, Tl, Ce, Co, Cu, Fe, Ga, Ge, Ir, Mo, Rh, Ru, Sb, Se, Sn, Sr, Ti and Zn ions. That is Mn may include other suitable metal ions, provided that at least two metal ions M (i.e. a first metal ion M and second metal ion M, otherwise referred to as M¹ and M²) are independently selected from Ag, Al, Au, Ba, Bi, Tl, Ce, Co, Cu, Fe, Ga, Ge, Ir, Mo, Rh, Ru, Sb, Se, Sn, Sr, Ti and Zn ions and are different. Mn may for instance include one or more sodium ions. Alternatively, or additionally, other suitable metals for forming salts of the invention may be included within Mn, provided at least a first metal ion selected from Ag, Al, Au, Ba, Bi, Tl, Ce, Co, Cu, Fe, Ga, Ge, Ir, Mo, Rh, Ru, Sb, Se, Sn, Sr, Ti and Zn ions, and a second metal ion is selected from Ag, Al, Au, Ba, Bi, Tl, Ce, Co, Cu, Fe, Ga, Ge, Ir, Mo, Rh, Ru, Sb, Se, Sn, Sr, Ti and Zn ions wherein the first and second elements are different.

In embodiments of present invention, Mₙ may comprise at least one Ag ion and at least one Al ion. Mn may comprise at least one Ag ion and at least one Bi ion. Mn may comprise at least one Ag ion and at least one Cu ion. Mn may comprise at least one Ag ion and at least one Zn ion. Mn may comprise at least one Ag ion and at least one Mo ion. Mn may comprise at least one Cu ion and at least one Zn ion. Mₙ may comprise at least two Ag ions and at least one Al ion. Mn may comprise at least two Ag ions and at least one Bi ion. Mn may comprise at least two Ag ions and at least one Cu ion. Mn may comprise at least two Ag ions and at least one Zn ion. Mₙ may comprise at least three Ag ions and at least one Al ion. Mn may comprise at least three Ag ions and at least one Bi ion. Mₙ may comprise at least three Ag ions and at least one Cu ion. Mn may comprise at least three Ag ions and at least one Zn ion. In embodiments such as those listed above, Mn may consist of said metal ions, i.e. the listed metal ions may constitute all of the metal ions Mn. However, in embodiments wherein Mn contains further metal ions additional to the listed metal ions above (i.e. where n is an integer larger than the number of metal ions specifically listed in the embodiments above), the balance of metal ions may comprise, or consist of sodium. As an example, in the embodiment above wherein Mₙ comprises at least one Ag ion and at least one Al ion, it may be that only one Ag ion and only one Al ion is present. If n is 4 in such an embodiment, then there is a balance of 2 unnamed metal ions M in Mn. Such metal ions may be selected from suitable metals, such as monovalent metal ions, e.g. sodium. Other suitable “balancing” metal ions will be apparent to the skilled person.

### P Group

In accordance with the present invention and embodiments thereof, component P, in the formula Mₙ(P), is an aminopolycarboxyl component comprising an optionally substituted alkylene amino backbone containing from 3 to 5 nitrogen atoms in the backbone and 5 or 6 carboxyl groups appended to the backbone, wherein the aminopolycarboxyl component contains from 10 to 20 atoms in the longest linear chain. The presence of substituents on the alkylene amino backbone is optional. Thus, it may be that the alkylene amino backbone is not substituted.

It will be appreciated that the term “aminopolycarboxyl component” as mentioned herein refers to any chemical moiety containing at least one amino group and two or more carboxyl groups. It will be understood that one or more of the carboxyl groups within the P group may form a carboxylate (-COO⁻) species during the formation of the compound of formula Mₙ(P). In this situation, the carboxyl group will include a negative charge (and may thus be capable of ionic bonding with a metal cation M). In certain embodiments, aminopolycarboxyl component may be a chelating moiety capable of coordinating to two or more sites of a single metal ion when forming part of a compound of formula Mₙ(P).

As referred to herein, “optionally substituted alkylene amino backbone” refers to a chemical moiety containing at least one optionally substituted alkylene group connected to at least one amino group to provide a core or main chain from which pendant carboxyl groups may be appended, or any respective substituents, if present.

In aspects and embodiments described herein, the optionally substituted alkylene amino backbone may be an optionally substituted C₁₋₃alkylene amino backbone. Preferably, the optionally substituted alkylene amino backbone contains optionally substituted methylene amino groups, optionally substituted ethylene amino groups or optionally substituted propylene amino groups. It is preferred that P include optionally substituted ethylene amino groups in the backbone. An especially preferred example of a P group containing ethylene amino groups in the backbone is DTPA or TTHA, as described herein. In embodiments, the backbone is not substituted, i.e. does not contain additional substituents.

In accordance with aspects and embodiments described herein, the optionally substituted alkylene amino backbone may contain from 3 to 5 nitrogen atoms in the backbone. In some embodiments, the optionally substituted alkylene amino backbone may contain 3, 4, or 5 nitrogen atoms in the backbone, typically, the optionally substituted alkylene amino backbone contains 3 or 4 nitrogen atoms in the backbone.

In accordance with aspects and embodiments described herein, the aminopolycarboxyl component may contain 5 or 6 carboxyl groups appended to the backbone. In further embodiments, the aminopolycarboxyl component may contain 3 nitrogen atoms in the backbone and 5 carboxyl groups. Alternatively, the aminopolycarboxyl component may contain 4 nitrogen atoms and 6 carboxyl groups.

It will be appreciated that a given carboxyl group may appended to the backbone via a direct covalent bond between the carbon atom of the carboxyl group and a suitable atom located within the backbone or via any suitable linking group capable of attaching the carbon atom of the carboxyl group to a suitable atom located within the backbone. Suitable linking groups will be readily apparent to the skilled person and, typically, may include optionally substituted -C₁₋₃alkylene groups, preferably methylene groups.

In accordance with aspects and embodiments described herein the aminopolycarboxyl component contains from 10 to 20 atoms in the longest linear chain. In embodiments, the aminopolycarboxyl component contains from 11 to 20 atoms in the longest linear chain, from 12 to 20 atoms in the longest linear chain, from 13 to 20 atoms in the longest linear chain, from 14 to 20 atoms in the longest linear chain, from 15 to 20 atoms in the longest linear chain, from 16 to 20 atoms in the longest linear chain, from 17 to 20 atoms in the longest linear chain or from 18 to 20 atoms in the longest linear chain. In other embodiments, the aminopolycarboxyl component contains from 10 to 19 atoms in the longest linear chain, from 10 to 18 atoms in the longest linear chain, from 10 to 17 atoms in the longest linear chain, from 10 to 16 atoms in the longest linear chain, from 10 to 15 atoms in the longest linear chain, from 10 to 14 atoms in the longest linear chain, from 10 to 13 atoms in the longest linear chain, from 10 to 12 atoms in the longest linear chain or from 10 to 11 atoms in the longest linear chain. In embodiments, the aminopolycarboxyl component contains 10 atoms in the longest linear chain, 11 atoms in the longest linear chain, 12 atoms in the longest linear chain, 13 atoms in the longest linear chain, 14 atoms in the longest linear chain, 15 atoms in the longest linear chain, 16 atoms in the longest linear chain, 17 atoms in the longest linear chain, 18 atoms in the longest linear chain, 19 atoms in the longest linear chain or 20 atoms in the longest linear chain. In embodiments, the aminopolycarboxyl component contains from 11 to 19 atoms in the longest linear chain, from 12 to 18 atoms in the longest linear chain, from 13 to 17 atoms in the longest linear chain, from 14 to 16 atoms in the longest linear chain. Preferably, the aminopolycarboxyl component contains from to 12 to 18 atoms in the longest linear chain or from 13 to 17 atoms in the longest linear chain.

It is intended that the longest linear chain does not include hydrogen atoms within the chain (but includes heteroatoms and carbons). For example, where P is based on DTPA, the longest linear chain will contain 13 atoms wherein the chain is counted from the oxygen atom forming part of the hydroxyl group within the terminal carboxyl group which is located at a first end of the DPTA molecule to the oxygen atom forming part of the hydroxyl group within the terminal carboxyl group which is located at the end of the DPTA molecule is furthest from the first end.

In embodiments of the present invention, P is a component or compound according to formula (I):

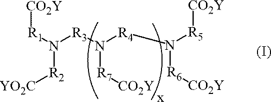

wherein X is 1 or 2 and each Y group is independently H or a negative charge wherein at least two Y groups are negative charges; and R₁, R₂, R₃, R₄, R₅, R₆ and R₇ are each independently optionally substituted C₁₋₃alkylene.

In accordance with any of the aspects or embodiments described herein, X is 1 or 2. Preferably, X is 1.

In accordance with any of the aspects or embodiments described herein, each Y group is independently H or a negative charge wherein at least two Y groups are negative charges. It will be appreciated that, when a given Y group is a negative charge, the carboxyl group (i.e. -CO₂Y), which forms part of the Y group, may chelate or coordinate to a suitable metal ion M within the compound of formula Mₙ(P). It is also envisaged that, when a given Y group is a negative charge, the carboxyl group (i.e. -CO₂Y) which forms part of the Y group could form an ionic bond to a suitable metal ion M within the compound of formula Mₙ(P).

In accordance with any of the aspects or embodiments described herein, R₁, R₂, R₃, R₄, R₅, R₆ and R₇ are each independently optionally substituted C₁₋₃alkylene. In some embodiments, R₁, R₂, R₃, R₄, R₅, R₆ and R₇ are each independently selected from optionally substituted methylene, optionally substituted ethylene and optionally substituted propylene groups. Typically, R₃ and R₄ are each optionally substituted ethylene and/or wherein R₁, R₂, R₅, R₆ and R₇ are each independently optionally substituted methylene or ethylene. In other embodiments, R₃ and R₄ are each optionally substituted ethylene and R₁, R₂, R₅, R₆ and R₇ are each optionally substituted methylene. Preferably, R₃ and R₄ are each ethylene and R₁, R₂, R₅, R₆ and R₇ are each methylene.

In accordance with preferred embodiments of the present invention, P may be a carboxylate of diethylenetriaminepentaacetic acid (“DTPA”) or triethylenetetramine-N,N,N′,N″,N‴,N‴-hexaacetic acid (“TTHA”). In this regard, it will be appreciated that the aminopolycarboxylic acid component P in such compounds is a carboxylate anion of DTPA or TTHA. Typically, at least two carboxylic acid functionalities of parent compound are present as the carboxylate. Thus, the compound of formula Mₙ(P) may preferably be Mₙ(DTPA) or Mₙ(TTHA), wherein the definition of Mₙ is as defined according to the first aspect or any embodiment thereof as described herein. The compound of formula Mₙ(P) may thus be Mₙ(DTPA) (with Mₙ as described herein). The compound of formula Mₙ(P) may be Mₙ(TTHA), with Mₙ as described herein.

### Specific Compounds

In accordance with any of the aspects or embodiments described herein, the compound of the formula Mₙ(P) may be selected from AgCu(DPTA), AgZn(DPTA), AgAI(DPTA), AgBi(DPTA), AgMo(DPTA), AgSr(DPTA), Ag₂Cu(DPTA), Ag₂Zn(DPTA), Ag₂Al(DPTA), Ag₂Bi(DPTA), Ag₃Cu(DPTA), Ag₃Zn(DPTA), AgCu(TTHA), AgZn(TTHA), AgAl(TTHA), AgBi(TTHA), AgMo(TTHA), AgSr(TTHA) Ag₂Cu(TTHA), Ag₂Zn(TTHA), Ag₂Al(TTHA), Ag₂Bi(TTHA), Ag₃Cu(TTHA), Ag₃Zn(TTHA), Ag₃Al(TTHA) Ag₃Bi(TTHA), Ag₄Cu(TTHA) and Ag₄Zn(TTHA). The compound of the formula Mₙ(P) may be selected from AgCu(DPTA), AgZn(DPTA), AgAl(DPTA), AgBi(DPTA), Ag₂Cu(DPTA), Ag₂Zn(DPTA), Ag₂Al(DPTA) and Ag₂Bi(DPTA). In preferred embodiments of the present invention, the compound of formula Mₙ(P) may be selected from AgCu(DPTA), AgCu(TTHA), AgZn(DPTA), AgZn(TTHA), Ag₂Al(DPTA), Ag₂Al(TTHA), AgBi(DPTA) and AgBi(TTHA). The DTPA compounds above are particularly preferred.

### Substituents

Where groups of the invention are described as being “optionally substituted” groups (e.g. optionally substituted alkylene amino backbone or optionally substituted -C₁₋₃alkylene) the respective group may be substituted or unsubstituted, for instance unsubstituted. Typically, substitution is intended to mean the notional replacement of a hydrogen atom with a substituent group, or two hydrogen atoms in the case of substitution by =O.

Where substituents are present, there may, for instance, be from 1 to 6 substituents, depending on the available substituent positions of the group. Typically, there will be from 1 to 3 substituents, in embodiments 1 or 2 substituents, such as only 1 substituent.

In such embodiments, the optional substituent(s) may each independently be -OH, =O, halo, -C₁₋₆alkyl, -C₂₋₆heteroalkyl, -C₃₋₆cycloalkyl, -C₃₋₆heterocycloalkyl, -C₂₋₆alkenyl, -C₂₋₆heteroalkenyl, -C₃₋₆cycloalkenyl, -C3-6heterocycloalkenyl, -C₂₋₆alkynyl, -C₂₋₆heteroalkynyl, halogen, or -C₁₋₆haloalkyl.

In other embodiments, the optional substituent(s) is/are each independently -C₁₋₆alkyl, -C₂₋₆heteroalkyl, halo, -C₁₋₆haloalkyl, or =O. The optional substituents may each independently be halo, e.g. independently selected from F, Cl, Br and I.

### Chemical Groups

### Halo

The term “halogen” (or “halo”) includes fluorine, chlorine, bromine and iodine.

### Alkyl, Alkylene, Alkenyl, Alkynyl, Cycloalkyl Etc.

The terms “alkyl”, “alkylene”, “alkenyl” or “alkynyl” are used herein to refer to both straight and branched chain acyclic forms. Cyclic analogues thereof are referred to as cycloalkyl, etc.

The term “alkyl” includes monovalent, straight or branched, saturated, acyclic hydrocarbyl groups. In embodiments, alkyl is methyl, ethyl, n-propyl, i-propyl or t-butyl groups.

The term “cycloalkyl” includes monovalent, saturated, cyclic hydrocarbyl groups.

The term “haloalkyl” refers to an alkyl group wherein at least one H is replaced by a halo group. In embodiments, haloalkyl refers to substitution by from 1-3 halo groups, e.g. 1. Examples include trihalomethyl, trihaloethyl, e.g. trifluoromethyl, etc.

The term “alkenyl” includes monovalent, straight or branched, unsaturated, acyclic hydrocarbyl groups having at least one carbon-carbon double bond and, in one embodiment, no carbon-carbon triple bonds.

The term “cycloalkenyl” includes monovalent, partially unsaturated, cyclic hydrocarbyl groups having at least one carbon-carbon double bond and, in one embodiment, no carbon-carbon triple bonds.

The term “alkynyl” includes monovalent, straight or branched, unsaturated, acyclic hydrocarbyl groups having at least one carbon-carbon triple bond and, in one embodiment, no carbon-carbon double bonds.

The term “alkylene” includes divalent, straight or branched, saturated, acyclic hydrocarbyl groups. In one embodiment alkylene is methylene, ethylene, n-propylene or i-propylene.

The term “alkenylene” includes divalent, straight or branched, unsaturated, acyclic hydrocarbyl groups having at least one carbon-carbon double bond and, in one embodiment, no carbon-carbon triple bonds.

### Heteroalkyl, Etc

The term “heteroalkyl” includes alkyl groups in which up to three carbon atoms, in one embodiment up to two carbon atoms, in another embodiment one carbon atom, are each replaced independently by O, S(O)ₜ or N, provided at least one of the alkyl carbon atoms remains. The heteroalkyl group may be C-linked or hetero-linked, i.e. it may be linked to the remainder of the molecule through a carbon atom or through O, S(O)ₜ or N, wherein t is defined below.

The term “heterocycloalkyl” includes cycloalkyl groups in which up to three carbon atoms, in one embodiment up to two carbon atoms, in another embodiment one carbon atom, are each replaced independently by O, S(O)ₜ or N provided at least one of the cycloalkyl carbon atoms remains. Examples of heterocycloalkyl groups include oxiranyl, thiaranyl, aziridinyl, oxetanyl, thiatanyl, azetidinyl, tetrahydrofuranyl, tetrahydrothiophenyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepariyl, thiepanyl, azepanyl, 1,4-dioxepanyl, 1,4-oxathiepanyl, 1,4-oxaazepanyl, 1,4-dithiepanyl, 1,4-thieazepanyl and 1,4-diazepanyl. The heterocycloalkyl group may be C-linked or N-linked, i.e. it may be linked to the remainder of the molecule through a carbon atom or through a nitrogen atom.

The term “heteroalkenyl” includes alkenyl groups in which up to three carbon atoms, in one embodiment up to two carbon atoms, in another embodiment one carbon atom, are each replaced independently by O, S(O)ₜ or N provided at least one of the alkenyl carbon atoms remains. The heteroalkenyl group may be C-linked or hetero-linked, i.e. it may be linked to the remainder of the molecule through a carbon atom or through O, S(O)ₜ or N.

The term “heterocycloalkenyl” includes cycloalkenyl groups in which up to three carbon atoms, in one embodiment up to two carbon atoms, in another embodiment one carbon atom, are each replaced independently by O, S(O)ₜ or N, provided at least one of the cycloalkenyl carbon atoms remains. Examples of heterocycloalkenyl groups include 3,4-dihydro-2H-pyranyl, 5-6-dihydro-2H-pyranyl, 2H-pyranyl, 1,2,3,4-tetrahydropyridinyl and 1,2,5,6-tetrahydropyridinyl. The heterocycloalkenyl group may be C-linked or N-linked, i.e. it may be linked to the remainder of the molecule through a carbon atom or through a nitrogen atom.

The term “heteroalkynyl” includes alkynyl groups in which up to three carbon atoms, in one embodiment up to two carbon atoms, in another embodiment one carbon atom, are each replaced independently by O, S(O)ₜ or N, provided at least one of the alkynyl carbon atoms remains. The heteroalkynyl group may be C-linked or hetero-linked, i.e. it may be linked to the remainder of the molecule through a carbon atom or through O, S(O)ₜ or N.

The term “heteroalkylene” includes alkylene groups in which up to three carbon atoms, in one embodiment up to two carbon atoms, in another embodiment one carbon atom, are each replaced independently by O, S(O)ₜ or N, provided at least one of the alkylene carbon atoms remains.

The term “heteroalkenylene” includes alkenylene groups in which up to three carbon atoms, in one embodiment up to two carbon atoms, in another embodiment one carbon atom, are each replaced independently by O, S(O)ₜ or N, provided at least one of the alkenylene carbon atoms remains.

Where mentioned above, t is independently 0, 1 or 2, for example 2. Typically, t is 0.

### Compositions of the Present Invention

The compounds of the present invention may be provided as a component in a composition, e.g. together with one or more suitable excipients. If the composition is intended for application to a human or animal body (e.g. during uses described herein), then it will be appreciated that the excipient should be a pharmaceutically acceptable excipient. Thus, the present disclosure provides compositions as described herein in accordance with the second and fifth aspects of the invention.

The composition of the second aspect comprises one or more compounds according to the first aspect of the present invention and any embodiment thereof described herein, and optionally further including one or more pharmaceutically acceptable excipients. According to the fifth aspect of the present invention, there is an anti-microbial composition obtainable by the method according to the fourth aspect of the present invention and any embodiment thereof described herein. Unless specified otherwise, the term composition as used herein refers to any compositions containing the compounds of the invention and may thus be applied to the context of compositions of the second aspect of the present invention as well as anti-microbial compositions of the fifth aspect.

The compound of formula Mn(P) in the compositions of the invention may have any of the suitable features and/or advantageous properties described herein in relation to the compounds of the first aspect. In addition, the composition of the fifth aspect of the present invention may have any of the suitable features and/or advantageous properties described herein in relation to the fourth aspect.

The composition of the second and fifth aspects of the present invention may be provided in any suitable form, such as in the form of a solid or liquid. The composition may be a solution or a suspension, or in a solid form, e.g. powder or tablet. In some embodiments, the composition is a fluid composition.

The composition may be a solution (e.g. aqueous solution), a lotion, an ointment, a cream, a balm, a gel, a paste or a solid, suitably powdered, composition.

In some embodiments, the composition of the second aspect of the present invention is a solution comprising the compound of formula Mₙ(P). In such solutions, the compound of formula Mₙ(P) is dissolved in a suitable solvent. The solution may comprise an aqueous solution such as water or saline, or another biocompatible solution in which the compound of formula Mₙ(P) is soluble. The solution may comprise an alcohol, for example ethanol.

In further embodiments, the composition of the second aspect of the present invention is a solution of the compound of formula Mₙ(P) in a solvent comprising water and ethanol, e.g. a mixture of water and ethanol. Such solutions may be highly efficacious and may be prepared by making a concentrated stock solution of the compound of formula Mₙ(P) in water and then introducing the desired concentration of ethanol. The solution of the compound of formula Mₙ(P) is preferably provided in a sterile and non-pyrogenic form and may be packaged in any convenient fashion. In some embodiments, the solution of the compound of formula Mₙ(P) may be provided in connection with or as part of a medical device, such as in a pre-filled syringe or another medical device. In some embodiments, the solution of the compound of the formula Mₙ(P) may be used as a catheter lock and flush solution. Such solutions are trapped or locked in a catheter whilst the catheter is not in use. In other embodiments, the solution of the compound of the formula Mₙ(P) may be used in a other lock devices which may, for example, form part of any suitable medical apparatus. Moreover, the solution of the compound of the formula Mₙ(P) may be used as a medical irrigation solution suitable for cleansing wounds or surgical sites (e.g. a mastitis dip solution or an ophthalmic solution) or an endodontic irrigation solution for cleaning/disinfecting dental water lines.

In further embodiments, the composition of the fifth aspect of the present invention is a solution of the compound obtainable by the method of the fourth aspect of the present invention in a mixture of water and ethanol. Such solutions may be highly efficacious and may be prepared by making a concentrated stock solution of the compound obtainable by the method of the fourth aspect of the present invention in water and then introducing the desired concentration of ethanol. The solution of the compound obtainable by the method of the fourth aspect of the present invention is preferably provided in a sterile and non-pyrogenic form and may be packaged in any convenient fashion. In some embodiments, the solution of the compound obtainable by the method of the fourth aspect of the present invention may be provided in connection with or as part of a medical device, such as in a pre-filled syringe or another medical device. In some embodiments, the compound obtainable by the method of the fourth aspect of the present invention may be used as a catheter lock and flush solution. Such solutions are trapped or locked in a catheter whilst the catheter is not in use. In other embodiments, the solution of the compound of the formula Mₙ(P) may be used in a other lock devices which may, for example, form part of any suitable medical apparatus. Moreover, the solution of the compound obtainable by the method of the fourth aspect of the present invention may be used as a medical irrigation solution suitable for cleansing wounds or surgical sites (e.g. a mastitis dip solution or an ophthalmic solution) or an endodontic irrigation solution for cleaning/disinfecting dental water lines.

The solution of the compound of formula Mₙ(P) according to embodiments of the second aspect or the solution of the compound obtainable by the method of the fourth aspect of the present invention may be prepared under sterile, aseptic conditions, or may be sterilized following preparation and/or packaging using any of a variety of suitable sterilization techniques. Single use vials, syringes or containers of the solution of the compound of formula Mₙ(P) or the solution of the compound obtainable by the method of the fourth aspect of the present invention may be provided. Multiple use vials, syringes or containers may also be provided.

It will be appreciated that, when the compound of formula Mₙ(P) as described herein, or the compound obtainable by the method of the fourth aspect of the present invention is present in a solution, it is possible to evenly coat a suitable surface/substrate (e.g. medical device or wound site) using established methods such as dipping, spraying and/or padding.

In some embodiments, compositions according to the invention may be an aqueous solution. Aqueous solutions contain water as a solvent, and in preferred embodiments contain mostly water by volume as a solvent, and in embodiments may contain only water as the liquid solvent in the solution.

In embodiments, the pH of the composition is no more than 12.5. In some embodiments, the pH of the composition is no less than 2. Suitably the pH of the composition is in the range from 2.0 to 12.0, suitably in the range from 4.0 to 10.0, suitably from 4.5 to 8.0. Suitably the pH of the composition is in the range from 4.0 to 7.0, suitably in the range from 4.0 to 6.0, suitably from 4.0 to 5.0.

In embodiments, the composition is an aqueous solution having a pH in the range from 4.0 to 12.0, suitably in the range from 4.0 to 11.0, suitably in the range from 4.0 to 10.0, suitably from 4.5 to 8.0. Suitably the pH of the composition is in the range from 4.0 to 7.0, suitably in the range from 4.0 to 6.0, suitably from 4.0 to 5.0.

In embodiments, the composition is provided in a solid form, such as a substantially dry form. Such solid forms may be provided as a coating on a surface of a substrate, e.g. a conduit (such as tubing), or a medical or industrial device such as a catheter or a container and the like. Such solid forms (e.g. substantially dry forms) of the composition of this second aspect may be provided in a powder or lyophilized form that may be reconstituted to form a solution with the addition of a solvent. Solid forms (e.g. substantially dry forms) of the composition may be provided as a coating, or may be incorporated in a gel or another type of carrier, or encapsulated or otherwise packaged and provided on a surface as a coating or in a container. Such solid forms (e.g. substantially dry forms) of the compositions of the invention are formulated such that in the presence of a solvent, the composition forms a solution or suspension of the respective compound of the invention.

In certain embodiments, different encapsulation or storage techniques may be employed such that effective time release of the active compound of the invention is accomplished upon extended exposure to solutions. In this embodiment, the substantially dry composition of this second aspect may provide anti-microbial and/or anti-biofilm and/or anti-inflammatory activity over an extended period of time and/or upon multiple exposures to solutions.

The compounds of the invention as provided in the compositions of the invention described herein, e.g. as present in the compositions of the second and fifth aspects described herein, may be present in the following amounts. The compound may be in an amount of at least 0.01 ppm, suitably at least 0.1 ppm, suitably at least 1.0 ppm, suitably at least 10 ppm, suitably at least 100 ppm, suitably at least 1,000 ppm, suitably at least 5,000 ppm. Suitably the compound is present in the composition in an amount of up to 100,000 ppm, suitably up to 10,000 ppm, suitably up to 5,000 ppm. Suitably the compound is present in the composition in an amount of from 0.01 ppm to 100,000 ppm, suitably from 0.1 ppm to 10,000 ppm, suitably from 1.0 ppm to 10,000 ppm, suitably from 10 ppm to 100 ppm, suitably from 100 ppm to 10,000 ppm, suitably from 1,000 ppm to 10,000 ppm.

Compositions according to the invention typically comprise a carrier and/or an excipient, suitably a pharmaceutically acceptable carrier and/or excipient. Suitable carriers and/or an excipients may be selected from water, ethanol, polypropylene glycol, glycerol, sorbitol, hydrocolloids, polyoxyethtylene block copolymers, carboxy methyl cellulose, pluronic F-127, cotton, chitosan, silicone, polyurethanes, acrylics, hydrogels, bamboo, soya, oils/fats, micelles, emulsions, paints, sodium alginate, polyethylene glycol, thickening agents such as Carbopol™ and mixtures thereof.

The compositions of the present invention may be in the form of a water-based gel. Suitably the composition may be a hydrogel which comprises one or more compounds of the invention, e.g. according to the first or fifth aspects of the invention. Suitably such hydrogels can maintain a moist wound healing environment and promote wound healing when said composition is applied to a wound, for example as part of a wound dressing. Such hydrogels may flow into the wound when applied to said wound to form an intimate contact with the wound bed and provide anti-microbial and/or anti-inflammatory effects to the whole wound. Suitably the hydrogel has a high enough viscosity that it does not flow out of wounds on areas of the body that are or become non-horizontal through movement of the patient. Suitably the hydrogel comprises a buffer, suitably to buffer the pH of the hydrogel to between 2.0 to 12.0, preferably, between 5.5 to 12.0. Suitable buffers are known in the art.

The composition may comprise a non-metal ion anti-microbial agent. For example, the composition may comprise quaternary ammonium compounds, iodine-based compounds and/or polyhexanides.

Suitable non-metal ion anti-microbial agents may be selected from any one or more of a electrolysed silane, electrolysed water, an antibiotic, halides (e.g. chlorine), benzylkonium chloride, chlorhexidine, a chlorhexadine salt, a triclosan, a polymoxin, a tetracycline, an amino glycoside (e.g. gentamicin or Tobramycin(TM)), a rifampicin, a bacitracin, an erythromycin, a neomycin, a chloramphenicol, a miconazole, a quinolone, a penicillin, a nonoxynol 9, a fusidic acid, a cephalosporin, a mupirocin, a metronidazole, a secropin, a protegrin, a bacteriocin, a defensin, a nitrofurazone, a mafenide, a acyclovir, a vanocmycin, a clindamycin, a lincomycin, a sulfonamide, a norfloxacin, a pefloxacin, a nalidizic acid, an oxalic acid, an enoxacin acid, a ciprofloxacin, a biguanide, iodine, tea tree oil, honey and superoxides. In one embodiment the anti-microbial agent comprises polyhexamethylene biguanide (PHMB) and/or derivatives thereof.

The non-metal ion anti-microbial agent may provide a beneficial anti-microbial which is additive to and/or synergistic with any anti-microbial effect provided by the compound of formula Mₙ(P).

In some embodiments, the composition of the present invention comprises polyhexamethylene biguanide (PHMB). For example, the composition may comprise Prontosan®. The compositions of the present invention may comprise polyhexamethylene biguanide in an amount of from about 0.01 % by weight of the overall composition to about 0.1 % by weight of the overall composition. Preferably, the present invention may comprise polyhexamethylene biguanide in an amount of from about 0.03 % by weight of the overall composition to about 0.08 % by weight of the overall composition, or about 0.05 % by weight of the overall composition.

In some embodiments, the composition of the present invention comprises hypochlorous acid. For example, the composition may comprise Suprox®. The compositions of the present invention may comprise hypochlorous acid in an amount of from about 0.01 % by weight of the overall composition to about 60 % by weight of the overall composition. Preferably, the present invention may comprise hypochlorous acid in an amount of from about 25 % by weight of the overall composition to about 50 % by weight of the overall composition, about 25 % by weight of the overall composition or about 50 % by weight of the overall composition.

In some embodiments, the compositions of the present invention comprises iodine (e.g. molecular iodine. The compositions of the present invention may comprise iodine in an amount of from about 0.01 % by weight of the overall composition to about 0.1 % by weight of the overall composition. Preferably, the present invention may comprise iodine in an amount of from about 0.03 % by weight of the overall composition to about 0.08 % by weight of the overall composition, or about 0.05 % by weight of the overall composition.

The compositions of the present invention may comprise a surfactant. A suitable surfactant may be sodium hexametaphosphate or a quaternary ammonium compound. A surfactant may improve the anti-biofilm effectiveness of the composition by removing matter from a biofilm during and/or after the action of the compound of the invention, e.g. compound according to formula Mₙ(P), or the compound obtainable by the method of the fourth aspect of the present invention, to disrupt the biofilm. For example, the compounds of the invention may act to disrupt the biofilm and, in doing so, produce flocculated cells. The surfactant may act to at least partially solubilise and remove these cells and prevent them from re-adhering to the biofilm and in doing so assist with the break-up and removal of the biofilm.

The composition may also comprise agents to improve wettability (e.g. poloxamers), stabilising agents, detergents and/or colour agents.

The compositions of the present invention may comprise an anti-biofilm agent other than the compounds of the invention and/or any non-metal ion anti-microbial agent, if present, and/or any surfactant, if present.

The composition may comprise an anti-biofilm agent selected from any one or more of DisperinB, DNase 1, ethylene glycol tetraacetic acid (EGTA), Proteinase K, apyrase, cis-2-decenoic acid, alginate lyase, lactoferrin, gallium, cellulose, citric acid, hexametaphosphate, quorum sensing blockers (e.g. Ql peptides), qorum sensing blockers and activators (e.g. cis-2-decenoic acid) and 5-fluorouracil, or EDTA. In some embodiments of compositions of the invention, EDTA is not present.

The composition of the present invention may comprise a transport enhancer compound. Such compounds are used to transport active ingredients across skin barrier for transdermal delivery and/or across individual cell membrane such as cell penetrating peptides (CPP) or glucose transporters. In some embodiment, the transport enhancer is dimethylsulphone or dimethylsulphoxide.

In some embodiments, the composition of the present invention comprises Tris (tris(hydroxymethyl)aminomethane). The compositions of the present invention may comprise Tris in an amount of from about 0.01 % by weight of the overall composition to about 10 % by weight of the overall composition. Preferably, the present invention may comprise Tris in an amount of from about 1 % by weight of the overall composition to about 5 % by weight of the overall composition, about 1.5 % by weight of the overall composition or about 3 % by weight of the overall composition.

In some embodiments, the composition of the present invention comprises urea. The compositions of the present invention may comprise urea in an amount of from about 0.01 % by weight of the overall composition to about 10 % by weight of the overall composition. Preferably, the present invention may comprise urea in an amount of from about 1 % by weight of the overall composition to about 5 % by weight of the overall composition, about 1.5 % by weight of the overall composition or about 3 % by weight of the overall composition.

In some embodiments, the composition of the present invention comprises taurine (2-aminoethanesulfonic acid). The compositions of the present invention may comprise taurine in an amount of from about 0.01 % by weight of the overall composition to about 10 % by weight of the overall composition. Preferably, the present invention may comprise taurine in an amount of from about 1 % by weight of the overall composition to about 5 % by weight of the overall composition, about 1.5 % by weight of the overall composition or about 3 % by weight of the overall composition.

The composition may comprise an anti-adhesion agent, for example, minocycline and/or rafampin.

The composition may comprise acids which themselves serve as antimicrobial agents, and can potentiate other antimicrobial compositions. Such acids may include formic acid, acetic acid, peracetic acid, malic acid, tannic acid, lactic acid and/or citric acid, hypochlorous acid.

The compositions may also include a reactive oxygen species. Such reactive oxygen species may include chlorine, hydrogen peroxide, percarbonates, chlorine dioxide, nitric oxide, hypochlorites and/or ozone.

In some embodiments, the composition comprises fibres, which are in contact with the one or more compounds of the invention. For example, the one or more compounds may be provided on the surface of the fibres and / or wherein the one or more compounds are incorporated within the fibres. Such fibres may be fibres in, or for, a wound dressing.

Such compositions comprising fibres may be formed by impregnating the composition into and/or coating the composition onto fibres. Suitable fibres may be selected from natural fibres, synthetic fibres and combinations thereof. Suitable fibres may be selected from any one or more fibres of cellulose, alginates, cotton, chitosan, soya, bamboo, carboxymethylcellulose, Rayon, Nylon, acrylic, polyester, polyurethane, polyurethane foam and combinations thereof.

Such compositions comprising fibres may be incorporated into and/or used to form a wound dressing (woven or non-woven). Such a wound dressing may have the advantage that the one or more compounds present in the composition are delivered to the wound, producing the beneficial effects referred to in relation to the first aspect. For example, the wound dressing may exhibit any one or more of anti-microbial, anti-biofilm and anti-inflammatory activities in use and therefore promote wound healing whilst combatting infection, if present.

Compositions of the second and fifth aspects of the present invention may be used with, or further comprise, debriding agents suitable for breaking down slough and necrotic tissue. Such debriding agents include enzymes/proteolytic agents such as collagenases, gelatinates and amylases.

### Methods of Forming Compounds and Compositions of the Present Invention

In a third aspect, the invention provides a method of forming a compound of formula Mₙ(P) wherein n is an integer from 2 to 6, each M is independently a metal ion, Mₙ comprises at least two different metal ions selected from Ag, Al, Au, Ba, Bi, Tl, Ce, Co, Cu, Fe, Ga, Ge, Ir, Mo, Rh, Ru, Sb, Se, Sn, Sr, Ti and Zn ions, and P is an aminopolycarboxyl component comprising an optionally substituted alkylene amino backbone containing from 3 to 5 nitrogen atoms in the backbone and 5 or 6 carboxyl groups appended to the backbone, wherein the aminopolycarboxyl component contains from 10 to 20 atoms in the longest linear chain. The method involves providing a solution comprising P, contacting the solution comprising P with a first metal ion source comprising a first metal ion M selected from Ag, Al, Au, Ba, Bi, Tl, Ce, Co, Cu, Fe, Ga, Ge, Ir, Mo, Rh, Ru, Sb, Se, Sn, Sr, Ti and Zn ions, and a second metal ion source comprising a second metal ion M selected from Ag, Al, Au, Ba, Bi, Tl, Ce, Co, Cu, Fe, Ga, Ge, Ir, Mo, Rh, Ru, Sb, Se, Sn, Sr, Ti and Zn ions, to form the compound of formula Mₙ(P), wherein the first metal ion M and second metal ion are independently selected from Ag, Al, Au, Ba, Bi, Tl, Ce, Co, Cu, Fe, Ga, Ge, Ir, Mo, Rh, Ru, Sb, Se, Sn, Sr, Ti and Zn ions, and the first metal ion M is different to the second metal ion M.

In a fourth aspect, the invention provides a method of preparing an anti-microbial composition comprising at least two different metal ions selected from Ag, Al, Au, Ba, Bi, Tl, Ce, Co, Cu, Fe, Ga, Ge, Ir, Mo, Rh, Ru, Sb, Se, Sn, Sr, Ti and Zn ions and a component P, wherein P is an aminopolycarboxyl component comprising an optionally substituted alkylene amino backbone containing from 3 to 5 nitrogen atoms in the backbone and 5 or 6 carboxyl groups appended to the backbone, wherein the aminopolycarboxyl component contains from 10 to 20 atoms in the longest linear chain. The method includes providing a solution comprising component P, contacting the solution comprising component P with a first metal ion source comprising a first metal ion M selected from Ag, Al, Au, Ba, Bi, Tl, Ce, Co, Cu, Fe, Ga, Ir, Mo, Rh, Ru, Ti and Zn ions and a second metal ion source comprising a second metal ion M that is different to be the first metal ion M, the second metal ion M selected from Ag, Al, Au, Ba, Bi, Tl, Ce, Co, Cu, Fe, Ga, Ge, Ir, Mo, Rh, Ru, Sb, Se, Sn, Sr, Ti and Zn ions to form the anti-microbial composition, wherein the first metal ion M is different to the second metal ion M, wherein the molar ratio of the component P, first metal ion source and second metal ion source (P : first metal ion : second metal ion) is from about 1: 2: 2 to about 1 : 10 : 10 or from about 1 : 0.1 : 0.1 to about 1 : 10 : 10.

It will be appreciated that the compounds of the formula Mₙ(P) are described with reference to the definitions provided herein. In addition, wherein referred to in the present disclosure, the terms “P” and “component P” are used interchangeably and it will be understood that both terms refer to an aminopolycarboxyl component as described herein.

In embodiments of the third and fourth aspects of the present invention, the methods further comprise contacting the solution comprising P according to the third aspect, or the solution comprising component P according to the fourth aspect, with the first metal ion source comprising a first metal ion M to form a precursor solution and then contacting the precursor solution with a second metal ion source comprising a second metal ion M to form the compound of formula Mₙ(P) according to the third aspect or the anti-microbial composition according to the fourth aspect.

Where a precursor solution is formed, the precursor solution may comprise a solid containing an intermediate P complexed with the first metal ion and a liquid supernatant containing one or more by-products. In such embodiments, the method further comprises subjecting the precursor solution to a suitable separation step to separate the solid from the supernatant. Suitable separation techniques include, but are not limited to, subjecting the precursor solution to filtration and/or centrifugation. In such embodiments, it will be appreciated that, once separated, the intermediate P complexed with the first metal ion is then subjected to a step of contacting the second metal ion source comprising a second metal ion M to form the compound of formula Mₙ(P) according to the third aspect or the anti-microbial composition according to the fourth aspect.

In embodiments, the step of subjecting the precursor solution to centrifugation comprises performing the centrifugation at from 1000 rpm to about 4000 rpm, from 1500 rpm to about 4000 rpm, from 2000 rpm to about 4000 rpm, from 2500 rpm to about 4000 rpm, from 3000 rpm to about 4000 rpm, from 3500 rpm to about 4000 rpm, from 1000 rpm to about 3500 rpm, from 1000 rpm to about 3000 rpm, from 1000 rpm to about 2500 rpm, from 1000 rpm to about 2000 rpm or from 1000 rpm to about 1500 rpm. The step of subjecting the precursor solution to centrifugation may also comprises performing the centrifugation at from 1500 rpm to about 3500 rpm, from 2000 rpm to about 3000 rpm or about 2500 rpm. In such embodiments, the step of subjecting the precursor solution to centrifugation comprises performing the centrifugation for about 1 minute to about 30 minutes, from about 1 minute to about 10 minutes, from about 1 minute to about 5 minutes, from about 2 minute to about 4 minutes, or about 3 minutes.

In embodiments, the step of subjecting the precursor solution to centrifugation comprises performing the centrifugation at from 1500 rpm to about 3500 rpm, from 2000 rpm to about 3000 rpm or about 2500 rpm for about 1 minute to about 5 minutes. Typically, the step of subjecting the precursor solution to centrifugation comprises performing the centrifugation at about 2500 rpm for about 3 minutes.

In further embodiments of the third and fourth aspects of the present invention, the methods further comprise mixing the first metal ion source comprising the first metal ion M and the second metal ion source comprising the second metal ion M to form a mixed precursor solution comprising the first and second metal ions before then contacting the solution comprising P with the mixed precursor solution to form the compound of formula Mₙ(P) according to the third aspect or the anti-microbial composition according to the fourth aspect of the present invention.

In embodiments of the third and fourth aspect of the present invention, the solution comprising P is an aqueous solution containing P or an organic solution containing P. The aqueous solution containing P may be formed by dissolving P in any suitable aqueous solvent or solution. For example, in embodiments, the aqueous solution containing P may be formed by dissolving P in demineralised water or an aqueous sodium hydroxide (NaOH) solution. It will be understood that when P is dissolved in an aqueous sodium hydroxide solution formation of an aqueous sodium salt solution comprising the sodium salt of P may occur. Preferably, the aqueous sodium salt solution comprising the sodium salt of P is a DPTA sodium salt solution or a TTHA sodium salt solution.

In embodiments, the aqueous sodium hydroxide solution used to form the aqueous sodium salt solution comprising the sodium salt of P may be an aqueous sodium hydroxide solution of from 20% (w/w) to 60% (w/w), 25% (w/w) to 55% (w/w), 30% (w/w) to 50% (w/w) or 35% (w/w) to 45% (w/w).

In embodiments, the pH of the solution comprising P is up to 12.5. Suitably the pH of the solution comprising P is in the range from 4.0 to 12.0, suitably in the range from 4.0 to 10.0, suitably from 4.5 to 8.0. Suitably the pH of the solution comprising P is in the range from 4.0 to 7.0, suitably in the range from 4.0 to 6.0, suitably from 4.0 to 5.0. The pH of the solution comprising P is typically less than or equal to 10.0. Preferably, the solution comprising P is an aqueous solution comprising P wherein the pH of the aqueous solution comprising P is in the range from 4.0 to 12.0, from 4.0 to 10.0, from 4.5 to 8.0 from 6.0 to 10.0 or from 8.0 to 10.0.

In embodiments, the concentration of P in the solution comprising P is from about 0.1 mol/Kg to about 1.0 mol/Kg, from about 0.2 mol/Kg to about 1.0 mol/Kg, from about 0.3 mol/Kg to about 1.0 mol/Kg, from about 0.4 mol/Kg to about 1.0 mol/Kg, from about 0.5 mol/Kg to about 1.0 mol/Kg, from about 0.6 mol/Kg to about 1.0 mol/Kg, from about 0.7 mol/Kg to about 1.0 mol/Kg, from about 0.8 mol/Kg to about 1.0 mol/Kg or from about 0.9 mol/Kg to about 1.0 mol/Kg. In other embodiments, the concentration of P in the solution comprising P is from about 0.1 mol/Kg to about 0.9 mol/Kg, from about 0.1 mol/Kg to about 0.8 mol/Kg, from about 0.1 mol/Kg to about 0.7 mol/Kg, from about 0.1 mol/Kg to about 0.6 mol/Kg, from about 0.1 mol/Kg to about 0.5 mol/Kg, from about 0.1 mol/Kg to about 0.4 mol/Kg, from about 0.1 mol/Kg to about 0.3 mol/Kg or from about 0.1 mol/Kg to about 0.2 mol/Kg. In further embodiments, the concentration of P in the solution comprising P is from about 0.2 mol/Kg to about 0.8 mol/Kg or from about 0.4 mol/Kg to about 0.6 mol/Kg. Typically, the concentration of P in the solution comprising P is from about 0.5 mol/Kg to about 0.8 mol/Kg, preferably, from about 0.55 mol/Kg to about 0.75 mol/Kg or about 0.65 mol/Kg.

In embodiments of the third aspect, n may be 2, 3, 4, 5 or 6. Preferably, n is 2, 3 or 4.

In embodiments of the third and fourth aspects of the present invention, the optionally substituted alkylene amino backbone may be an optionally substituted -C₁₋₃alkylene amino backbone. Preferably, the optionally substituted alkylene amino backbone may be an optionally substituted methylene amino backbone.

The optionally substituted alkylene amino backbone may contain 3, 4, or 5 nitrogen atoms in the backbone, typically, the optionally substituted alkylene amino backbone contains 3 or 4 nitrogen atoms in the backbone. There may be 3 nitrogen atoms. There may be 4 nitrogen atoms.

The optionally substituted alkylene amino backbone may contain 3 nitrogen atoms and there may be 5 carboxyl groups appended to the backbone. Alternatively, the optionally substituted alkylene amino backbone may contain 4 nitrogen atoms and there may be 6 carboxyl groups appended to the backbone.

In further embodiments of the third and fourth aspects of the present invention, the aminopolycarboxyl component contains from 12 to 18 atoms in the longest linear chain or from 13 to 17 atoms in the longest linear chain.

In accordance with embodiments of the third and fourth aspects of the present invention, P may be a component or compound according to formula (I):

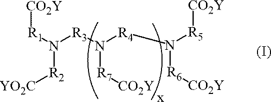

wherein X is 1 or 2 and each Y group is independently H or a negative charge wherein at least two Y groups are negative charges; and R₁, R₂, R₃, R₄, R₅, R₆ and R₇ are each independently optionally substituted -C₁₋₃alkylene.

In some embodiments of the third and fourth aspects of the present invention, X is 1.

In other embodiments of the third and fourth aspects of the present invention, R₃ and R₄ are each optionally substituted ethylene and/or wherein R₁, R₂, R₅, R₆ and R₇ are each optionally substituted methylene or ethylene. In other embodiments, R₃ and R₄ are each optionally substituted ethylene and R₁, R₂, R₅, R₆ and R₇ are each optionally substituted methylene. Preferably, R₃ and R₄ are each ethylene and R₁, R₂, R₅, R₆ and R₇ are each methylene.

In further embodiments of the present invention, P is DTPA or TTHA. Preferably, P is DTPA.

In embodiments of the third and fourth aspects of the present invention, the method further comprises contacting the solution comprising P with one to four further sources of metal ions M to form the compound of formula Mₙ(P) according to the third aspect or the anti-microbial composition according to the fourth aspect.

Typically, the first metal ion source may comprise a metal ion selected from Ag, Al, Au, Ba, Bi, Tl, Ce, Co, Cu, Fe, Ga, Ge, Ir, Mo, Rh, Ru, Sb, Se, Sn, Sr, Ti and Zn ions. The second metal ion source may comprise a metal ion selected from Ag, Al, Au, Ba, Bi, Tl, Ce, Co, Cu, Fe, Ga, Ge, Ir, Mo, Rh, Ru, Sb, Se, Sn, Sr, Ti and Zn ions. Furthermore, one to four further sources of metal ions may comprise a metal ion selected from Ag, Al, Au, Ba, Bi, Tl, Ce, Co, Cu, Fe, Ga, Ge, Ir, Mo, Rh, Ru, Sb, Se, Sn, Sr, Ti and Zn ions.

In the methods of the third and fourth aspects of the present invention, the first metal ion source may be an Ag metal ion source and the second metal ion source may be a Cu metal ion source. Alternatively, the first metal ion source may be a Cu metal ion source and the second metal ion source may be a Ag metal ion source. In some embodiments of the third and fourth aspects of the present invention, the first metal ion source may be an Ag metal ion source and the second metal ion source may be a Zn metal ion source. In embodiments of the third and fourth aspects of the present invention, the first metal ion source may be a Zn metal ion source and the second metal ion source may be a Ag metal ion source. In other embodiments of the third and fourth aspects of the present invention, the first metal ion source may be an Al metal ion source and the second metal ion source may be a Ag metal ion source. The first metal ion source may be a Ag metal ion source and the second metal ion source may be an Al metal ion source. In yet further embodiments of the third and fourth aspects of the present invention, the first metal ion source may be a Ag metal ion source and the second metal ion source may be a Bi metal ion source or the first metal ion source may be a Bi metal ion source and the second metal ion source may be an Ag metal ion source.

In embodiments of the third and fourth aspects of the present invention, the first metal ion, the second metal ion source or the one to four further metal ion sources may each independently be a solid or a solution. Where the any of the first metal ion source, the second metal ion source or the one to four further metal ion sources is a solution, the solution has a metal ion source concentration of from about 0.01 mol/Kg to about 10 mol/Kg, from about 0.01 mol/Kg to about 8 mol/Kg or from about 0.1 mol/Kg to about 5 mol/Kg. In some embodiments, where the any of the first metal ion source, the second metal ion source or the one to four further metal ion sources is a solution, the solution has a metal ion source concentration of from about 0.5 mol/Kg to about 7.5 mol/Kg, from about 0.5 mol/Kg to about 7.0 mol/Kg, from about 0.5 mol/Kg to about 6.5 mol/Kg, from about 0.5 mol/Kg to about 6.5 mol/Kg, from about 0.5 mol/Kg to about 6.0 mol/, from about 0.5 mol/Kg to about 5.5 mol/Kg, from about 0.5 mol/Kg to about 5.0 mol/Kg from about 0.5 mol/Kg to about 4.5 mol/Kg, from about 0.5 mol/Kg to about 4.0 mol/Kg or from about 0.5 mol/Kg to about 3.5 mol/Kg.

In embodiments of the third and fourth aspects of the present invention, where the first metal ion is a solution, the first metal ion source concentration is from about 0.01 mol/Kg to about 1.0 mol/Kg, from about 0.02 mol/Kg to about 1.0 mol/Kg, from about 0.03 mol/Kg to about 1.0 mol/Kg, from about 0.04 mol/Kg to about 1.0 mol/Kg, from about 0.05 mol/Kg to about 1.0 mol/Kg, from about 0.06 mol/Kg to about 1.0 mol/Kg, from about 0.07 mol/Kg to about 1.0 mol/Kg, from about 0.08 mol/Kg to about 1.0 mol/Kg, from about 0.09 mol/Kg to about 1.0 mol/Kg, from about 0.1 mol/Kg to about 1.0 mol/Kg, from about 0.2 mol/Kg to about 1.0 mol/Kg, from about 0.3 mol/Kg to about 1.0 mol/Kg, from about 0.4 mol/Kg to about 1.0 mol/Kg, from about 0.5 mol/Kg to about 1.0 mol/Kg, from about 0.6 mol/Kg to about 1.0 mol/Kg, from about 0.7 mol/Kg to about 1.0 mol/Kg, from about 0.8 mol/Kg to about 1.0 mol/Kg or from about 0.9 mol/Kg to about 1.0 mol/Kg. In other embodiments, the first metal ion source concentration is from is from about 0.1 mol/Kg to about 0.9 mol/Kg, from about 0.1 mol/Kg to about 0.8 mol/Kg, from about 0.1 mol/Kg to about 0.7 mol/Kg, from about 0.1 mol/Kg to about 0.6 mol/Kg, from about 0.1 mol/Kg to about 0.5 mol/Kg, from about 0.1 mol/Kg to about 0.4 mol/Kg, from about 0.1 mol/Kg to about 0.3 mol/Kg or from about 0.1 mol/Kg to about 0.2 mol/Kg. In further embodiments, the first metal ion source concentration from about 0.2 mol/Kg to about 0.8 mol/Kg or from about 0.4 mol/Kg to about 0.6 mol/Kg.

In embodiments of the third and fourth aspects of the present invention, where the second metal ion is a solution, the second metal ion source concentration is from about 0.01 mol/Kg to about 1.0 mol/Kg, from about 0.02 mol/Kg to about 1.0 mol/Kg, from about 0.03 mol/Kg to about 1.0 mol/Kg, from about 0.04 mol/Kg to about 1.0 mol/Kg, from about 0.05 mol/Kg to about 1.0 mol/Kg, from about 0.06 mol/Kg to about 1.0 mol/Kg, from about 0.07 mol/Kg to about 1.0 mol/Kg, from about 0.08 mol/Kg to about 1.0 mol/Kg, from about 0.09 mol/Kg to about 1.0 mol/Kg, from about 0.1 mol/Kg to about 1.0 mol/Kg, from about 0.2 mol/Kg to about 1.0 mol/Kg, from about 0.3 mol/Kg to about 1.0 mol/Kg, from about 0.4 mol/Kg to about 1.0 mol/Kg, from about 0.5 mol/Kg to about 1.0 mol/Kg, from about 0.6 mol/Kg to about 1.0 mol/Kg, from about 0.7 mol/Kg to about 1.0 mol/Kg, from about 0.8 mol/Kg to about 1.0 mol/Kg or from about 0.9 mol/Kg to about 1.0 mol/Kg. In other embodiments, the second metal ion source concentration is from is from about 0.1 mol/Kg to about 0.9 mol/Kg, from about 0.1 mol/Kg to about 0.8 mol/Kg, from about 0.1 mol/Kg to about 0.7 mol/Kg, from about 0.1 mol/Kg to about 0.6 mol/Kg, from about 0.1 mol/Kg to about 0.5 mol/Kg, from about 0.1 mol/Kg to about 0.4 mol/Kg, from about 0.1 mol/Kg to about 0.3 mol/Kg or from about 0.1 mol/Kg to about 0.2 mol/Kg. In further embodiments, the second metal ion source concentration is from about 0.2 mol/Kg to about 0.8 mol/Kg or from about 0.4 mol/Kg to about 0.6 mol/Kg.

In embodiments of the third and fourth aspects of the present invention, where the first metal ion is a solution, the first metal ion source concentration is from about 0.01 mol/Kg to about 7.5 mol/Kg, from about 1.0 mol/Kg to about 7.5 mol/Kg, from about 0.01 mol/Kg to about 5.0 mol/Kg from about 1.0 mol/Kg to about 5.0 mol/Kg, from about 2.0 mol/Kg to about 4.0 mol/Kg, from about 2.5 mol/Kg to about 4.5 mol/Kg or about 3.5 mol/Kg.

In embodiments of the third and fourth aspects of the present invention, where the second metal ion is a solution, the second metal ion source concentration is from about 0.01 mol/Kg to about 7.5 mol/Kg, from about 1.0 mol/Kg to about 7.5 mol/Kg, from about 0.01 mol/Kg to about 5.0 mol/Kg, from about 1.0 mol/Kg to about 5.0 mol/Kg, from about 2.0 mol/Kg to about 4.0 mol/Kg, from about 2.5 mol/Kg to about 4.5 mol/Kg or about 3.5 mol/Kg.

In embodiments of the third and fourth aspects of the present invention, a Ag metal ion source comprises any Ag-containing reagent suitable for providing Ag metal ions, typically, silver nitrate (AgNO₃). The Ag metal ion source may be a silver nitrate solution, preferably, an aqueous silver nitrate solution. The concentration of silver nitrate in the aqueous silver nitrate solution is typically from about 0.01 mol/Kg to about 7.5 mol/Kg, from about 1.0 mol/Kg to about 7.5 mol/Kg, from about 0.01 mol/Kg to about 5.0 mol/Kg,from about 1.0 mol/Kg to about 5.0 mol/Kg, from about 2.0 mol/Kg to about 4.0 mol/Kg, from about 2.5 mol/Kg to about 4.5 mol/Kg, about 3.5 mol/Kg, from about 2.5 mol/Kg to about 7.5 mol/Kg, from about 2.5 mol/Kg to about 7.0 mol/Kg, from about 4.5 mol/Kg to about 7.0 mol/Kg, from about 5.5 mol/Kg to about 7.0 mol/Kg or about 6.5 mol/Kg.

In embodiments of the third and fourth aspects of the present invention, a Cu metal ion source comprises any Cu containing reagent suitable for providing Cu metal ions, typically, copper sulphate (CuSO₄). The Cu metal ion source may be a copper sulphate solution, preferably, an aqueous copper sulphate solution. The concentration of copper sulphate in the aqueous copper sulphate solution is typically from about 0.2 mol/Kg to about 0.8 mol/Kg, from about 0.4 mol/Kg to about 0.6 mol/Kg, about 0.5 mol/Kg or about 0.55 mol/Kg.

In embodiments of the third and fourth aspects of the present invention, an Al metal ion source comprises any Al containing reagent suitable for providing Al metal ions, typically, aluminium sulphate hexadecahydrate (Al₂H₃₂O₂₈S₃). The Al metal ion source may be an aluminium sulphate solution, preferably, an aqueous aluminium sulphate solution. The concentration of aluminium sulphate in the aqueous aluminium sulphate solution is typically from about 0.05 mol/Kg to about 1.1 mol/Kg, from about 0.05 mol/Kg to about 0.5 mol/Kg or from about 0.1 mol/Kg to about 1.1 mol/Kg.

In embodiments of the third and fourth aspects of the present invention, a Bi metal ion source comprises any Bi containing reagent suitable for providing Bi metal ions, typically, bismuth nitrate (Bi(NO₃)₃). The Bi metal ion source may be a bismuth nitrate solution, preferably, an aqueous bismuth nitrate solution. The concentration of bismuth nitrate in the aqueous bismuth nitrate solution is typically from about 0.01 mol/Kg to about 1.0 mol/Kg or from about 0.02 mol/Kg to about 0.5 mol/Kg.

In embodiments of the third aspect of the present invention, the molar ratio of P, first metal ion source and second metal ion source (P : first metal ion : second metal ion) is from about 1 : 2 : 2 to about 1 : 10 : 10. According to the fourth aspect of the present invention, the molar ratio of P, first metal ion source and second metal ion source (P : first metal ion : second metal ion) is from about 1 : 2 : 2 to about 1 : 10 : 10. In further embodiments of the third and fourth aspects of the present invention, the molar ratio of P, first metal ion source and second metal ion source (P : first metal ion : second metal ion) is from about 1 : 2 : 2 to about 1 : 9 : 9, from about 1 : 2 : 2 to about 1 : 8 : 8, from about 1 : 2 : 2 to about 1 : 7 : 7, from about 1 : 2 : 2 to about 1 : 6 : 6, from about 1 : 2 : 2 to about 1 : 5 : 5, from about 1 : 2 : 2 to about 1 : 4 : 4, or from about 1 : 2 : 2 to about 1 : 3 : 3. In other embodiments of the third and fourth aspects of the present invention, the molar ratio of P, first metal ion source and second metal ion source (P : first metal ion : second metal ion) is from about 1 : 3 : 3 to about 1 : 10 : 10, from about 1 : 4 : 4 to about 1 : 10 : 10, from about 1 : 5 : 5 to about 1 : 10 : 10, from about 1 : 6 : 6 to about 1 : 10 : 10, from about 1 : 7 : 7 to about 1 : 10 : 10, from about 1 : 8 : 8 to about 1 : 10 : 10 or from about 1 : 9 : 9 to about 1 : 10 : 10. In some embodiments of the third and fourth aspects of the present invention, the molar ratio of P, first metal ion source and second metal ion source (P : first metal ion : second metal ion) is from about 1 : 0.1 :: 0.1 to about 1 : 10 : 10, typically, from about 1: 0.2 : 0.1 to about 1 : 6 : 6.

In embodiments of the third and fourth aspects of the present invention, the molar ratio of P, first metal ion source and second metal ion source (P : first metal ion : second metal ion) is from about 1 : 2 : 2 to about 1 : 6 : 6. In some embodiments, P is DPTA or TTHA, the first metal ion source is a Ag, Al, Bi, Cu or Zn metal ion source and the second metal ion source is a Ag, Al, Bi, Cu or Zn metal ion source wherein the molar ratio of P, first metal ion source and second metal ion source (P : first metal ion : second metal ion) is from about 1 : 0.1 : 0.1 to about 1 : 10 : 10, from about 1: 0.2 : 0.1 to about 1 : 6 : 6 or from about 1 : 2 : 2 to about 1 : 6 : 6. In embodiments, the P is DPTA, the first metal ion source is a Ag, Al, Bi, Cu or Zn metal ion source and the second metal ion source is a Ag, Al, Bi, Cu or Zn metal ion source wherein the molar ratio of P, first metal ion source and second metal ion source (P : first metal ion : second metal ion) is from about 1 : 0.1 : 0.1 to about 1 : 10 : 10, from about 1: 0.2 : 0.1 to about 1 : 6 : 6 or from about 1 : 2 : 2 to about 1 : 6 : 6. In some embodiments P is DPTA, the first metal ion source is Ag, Al, Bi, Cu or Zn metal ion source and the second metal ion source is a Ag, Al, Bi, Cu or Zn metal ion source wherein the molar ratio of P, first metal ion source and second metal ion source (P : first metal ion : second metal ion) is from about 1 : 0.1 : 0.1 to about 1 : 10 : 10, from about 1: 0.2 : 0.1 to about 1 : 6 : 6 or from about 1 : 2 : 2 to about 1 : 6 : 6. Typically, P is DPTA, the first metal ion source is a Ag metal ion source and the second metal ion source is a Al, Bi, Cu or Zn metal ion source wherein the molar ratio of P, first metal ion source and second metal ion source (P : first metal ion : second metal ion) is from about 1 : 0.1 : 0.1 to about 1 : 10: 10, from about 1: 0.2 : 0.1 to about 1 : 6: 6 or from about 1 : 2: 2 to about 1 : 6 : 3.

### Wound Dressings and Medical Devices

The invention provides a wound dressing or medical device comprising a compound according to the invention, e.g. according to the first aspect of the present invention or any embodiment thereof, a composition according to the invention (e.g. second aspect of the present invention or any embodiment thereof) or a composition according to the fifth aspect of the present invention or any embodiment thereof as described herein.

Suitably the wound dressing comprises the compound of formula Mₙ(P) or any composition described herein in a wound contact layer. The wound contact layer may have been impregnated, coated, dipped, laminated and/or sprayed with the compound of formula Mₙ(P) or composition. Alternatively or additionally the wound dressing may comprise the compound of formula Mₙ(P) or any composition described herein in an absorbant layer, e.g. provided in contact with a wound contact layer. Alternatively or additionally the wound dressing may comprise the compound of formula Mₙ(P) or any composition described herein in an adhesive which contacts the skin in use.

The medical device may be a catheter. In some embodiments, the medical device may be an intubation tube. The medical device may be a medical tube, a conduit, an intravascular device, an implanted medical device, a medical or veterinary instrument, a contact lens, an optical implant or a dental, orthodontic or periodontal device. Where the medical device is a dental device, this may include dental water lines or dental wash apparatus (e.g. for soaking dentures or toothbrushes).

Suitably the compound according to the first aspect or the composition according to the second or fifth aspects is a component of a coating, e.g. coated onto at least a part of a surface of the medical device, suitably a surface which is intended to contact a part of a patient’s body, in use. Methods of coating the compound or composition onto such a surface are known in the art.

Suitably the medical device has a reduced capacity for biofilm formation than a comparable medical device of the prior art which does not comprise such a compound or composition. The medical device may therefore reduce or substantially prevent infections caused by biofilm formation and pathogenic microorganism growth on the medical device.

### Medical Uses of the Compounds or Compositions of the Present Invention

In a seventh aspect, the present invention provides the use of the compound according to the first aspect of the present invention or any embodiment thereof, the composition according to the second aspect of the present invention or any embodiment thereof or a composition according to the fifth aspect of the present invention or any embodiment thereof as described herein to sanitise and/or substantially remove a biofilm from a substrate wherein the use excludes using the compound or composition in a method for treatment of the human or animal body by surgery or therapy.

In an eighth aspect, the present invention provides a compound according to the first aspect of the present invention or any embodiment thereof, a composition according to the second aspect of the present invention or any embodiment thereof or a composition according to the fifth aspect of the present invention or any embodiment thereof as described herein for use as a medicament.

In a ninth aspect, the present invention provides a compound according to the first aspect of the present invention or any embodiment thereof, a composition according to the second aspect of the present invention or any embodiment thereof or a composition according to the fifth aspect of the present invention or any embodiment thereof as described herein for use in a method of sanitising and/or substantially removing a biofilm from a substrate, optionally wherein the substrate is a wound on a human or animal body.

In a further aspect, the present invention provides the use of a compound according to the first aspect of the present invention or any embodiment thereof, a composition according to the second aspect of the present invention or any embodiment thereof or a composition according to the fifth aspect of the present invention or any embodiment thereof as described herein in the manufacture of a medicament for sanitising and/or substantially removing a biofilm from a substrate, optionally wherein the substrate is a wound on a human or animal body.

A tenth aspect of the present invention provides a compound according to the first aspect of the present invention or any embodiment thereof, a composition according to the second aspect of the present invention or any embodiment thereof or a composition according to the fifth aspect of the present invention or any embodiment thereof as described herein for use in a method of treating infections of cuts, bruises, surgical sites, lacerations, abrasions, punctures, incisions, gunshots, burns, pyoderma, atopic dermatitis, eczema, psoriasis, pressure ulcers, venous and artery leg ulcers, diabetic foot ulcers, cystic fibrosis (CF)-associated infections, mastitis, otitis, community or hospital acquired infections or food-borne diseases.

A further aspect of the present invention provides the use of a compound according to the first aspect of the present invention or any embodiment thereof, a composition according to the second aspect of the present invention or any embodiment thereof or a composition according to the fifth aspect of the present invention or any embodiment thereof as described herein in the manufacture of a medicament for treating infections of cuts, bruises, surgical sites, lacerations, abrasions, punctures, incisions, gunshots, burns, pyoderma, atopic dermatitis, eczema, pressure ulcers, venous and artery leg ulcers, diabetic foot ulcers, cystic fibrosis (CF)-associated infections, mastitis, otitis, community or hospital acquired infections or food-borne diseases.

Another aspect of the present invention provides a compound according to the first aspect of the present invention or any embodiment thereof, a composition according to the second aspect of the present invention or any embodiment thereof or a composition according to the fifth aspect of the present invention or any embodiment thereof as described herein for use in a method of treating skin, dental and/or nail diseases or disorders, for example, acne, athletes foot, dental caries, periodontitis, gingivitis, eczema, psoriasis, rosacea, cold sores and/or fungating nail conditions.

A further aspect of the present invention provides the use of a compound according to the first aspect of the present invention or any embodiment thereof, a composition according to the second aspect of the present invention or any embodiment thereof or a composition according to the fifth aspect of the present invention or any embodiment thereof as described herein in the manufacture of a medicament for treating skin, dental and/or nail diseases or disorders, for example, acne, athletes foot, dental caries, periodontitis, gingivitis, eczema, psoriasis, rosacea, cold sores and/or fungating nail conditions.

In an eleventh aspect of the present invention there is a method of sanitising and/or substantially removing a biofilm from a substrate comprising treating the substrate with a compound according to the first aspect of the present invention or any embodiment thereof, a composition according to the second aspect of the present invention or any embodiment thereof or a composition according to the fifth aspect of the present invention or any embodiment thereof as described herein. Optionally, the method excludes using the compound or composition in a method of treatment of the human or animal body by surgery or therapy.

In a twelfth aspect of the present invention there is a method of treating infections of cuts, bruises, surgical sites, lacerations, abrasions, punctures, incisions, gunshots, burns, pyoderma, atopic dermatitis, eczema, pressure ulcers, venous and artery leg ulcers, diabetic foot ulcers, cystic fibrosis (CF)-associated infections, mastitis, otitis, community or hospital acquired infections or food-borne diseases comprising treating a subject with a compound according to the first aspect of the present invention or any embodiment thereof, a composition according to the second aspect of the present invention or any embodiment thereof or a composition according to the fifth aspect of the present invention or any embodiment thereof as described herein to a subject (i.e. wherein the respective compound or composition is administered in a pharmaceutically effective amount).

In a further aspect of the present invention there is a method of treating skin, dental and/or nail diseases or disorders, for example, acne, athletes foot, dental caries, periodontitis, gingivitis, eczema, psoriasis, rosacea, cold sores and/or fungating nail conditions comprising treating a subject with a compound according to the first aspect of the present invention or any embodiment thereof, a composition according to the second aspect of the present invention or any embodiment thereof or a composition according to the fifth aspect of the present invention or any embodiment thereof as described herein to a subject (i.e. wherein the respective compound or composition is administered in a pharmaceutically effective amount).

In embodiments of any of the ninth or eleventh aspect of the present invention the substrate may be any surface where biofilm treatment and/or removal is required. The substrate may be a wound on a human or animal body, the wound being of any of the types described above. The substrate may also include any human touch surface or touch point where that surface is required to be kept substantially free of microorganisms, such as touch surfaces or touch points in hospitals. The substrate may be a surface that is submerged in water but prone to surface biofilm formation such as the hull on a ship, or turbine blade which can be prone to marine microbial growth. In addition, the substrate may include industrial pipework or air conditioning units/ducting where biofouling is a common problem and can impede efficiency.

Still further areas where the compounds may be useful is in surface coatings for any suitable object. For example, the compounds are envisaged to be useful in marine coatings, where aquatic fouling is a common problem whereby microbial growth on marine hulls increase hydrodynamic friction leading to increased drag hence decreased fuel efficiency.

In embodiments of seventh aspect of the present invention, the substrate may be a part of a medical device. In some embodiments, the substrate may be a part of food preparation and processing equipment or a food product, for example meat processing equipment or meat products.

Suitably the use and/or method at least disrupts and/or disperses the biofilm. Suitably the use and/or method increases the susceptibility of the biofilm and the microorganisms within it to attack by the compound of the first aspect and/or any metal ions released from the compound and/or any additional agents (such as non-metal ion anti-microbial agents and/or anti-biofilm agents) present in the composition according to the second or fifth aspects. Suitably the use and/or method completely removes the biofilm from the substrate. Suitably the use and/or method sanitises the substrate. Suitably the use and/or method completely removes the biofilm from the substrate and sanitises the substrate.

The use and/or method carried out on a wound may advantageously facilitate wound healing and/or treat infections and/or reduce inflammation.

The use and/or method carried out on a medical device may advantageously clean and/or sanitise the medical device and therefore prevent infections caused by medical devices comprising biofilms harbouring pathogenic microorganisms.

The use and/or method carried out on a food product may advantageously slow or preferably stop the growth of pathogenic microorganisms on the food product and therefore prevent spoilage of the food product and food poisoning which may result from ingesting such food contaminated with pathogenic microorganisms.

According to a thirteenth aspect of the present invention there is provided a kit comprising a compound according to the first aspect or a composition according to the second or fifth aspects and a medical device or wound dressing.

Suitably the kit may comprise a solution or suspension of the compound of formula Mₙ(P) or a composition of the present invention suitably contained in a pre-filled syringe or another medical device. Suitably the medical device may be a catheter or an intubation tube.

According to a fourteenth aspect of the present invention there is provided a use of a compound according to the first aspect or a composition according to the second or fifth aspects to coat at least a part of a medical device.

According to a further aspect of the present invention there is provided a use of a compound according to the first aspect or a composition according to the second or fifth aspect in a wound dressing.

According to a further aspect of the present invention there is provided a use of a compound according to the first aspect or a composition according to the second or fifth aspect in a method of water treatment.

According to a further aspect of the present invention there is provided a use of a compound according to the first aspect or a composition according to the second or fifth aspect in a method of food preparation or food processing.

According to a further aspect of the present invention there is provided a use of a compound according to the first aspect or a composition according to the second or fifth aspect in a dental procedure.

According to a further aspect of the present invention there is provided a compound according to the first aspect of the present invention or any embodiment thereof, a composition according to the second aspect of the present invention or any embodiment thereof or a composition according to the fifth aspect of the present invention or any embodiment thereof as described herein for use in dental procedure.

According to a further aspect of the present invention there is provided the use of a compound according to the first aspect of the present invention or any embodiment thereof, a composition according to the second aspect of the present invention or any embodiment thereof or a composition according to the fifth aspect of the present invention or any embodiment thereof as described herein for the manufacture of a medicament for a dental procedure.

According to a further aspect of the present invention there is provided a method of using a compound according to the first aspect of the present invention or any embodiment thereof, a composition according to the second aspect of the present invention or any embodiment thereof, or a composition according to the fifth aspect of the present invention or any embodiment thereof as described herein in a dental procedure.

### Terms and Expressions Used Herein

Throughout this specification, the term “comprising” or “comprises” means including the component(s) specified but not to the exclusion of the presence of other components.

The term “consisting essentially of” or “consists essentially of” means including the components specified but excluding other components except for materials present as impurities, unavoidable materials present as a result of processes used to provide the components, and components added for a purpose other than achieving the technical effect of the invention. Typically, when referring to compositions, a composition consisting essentially of a set of components will comprise less than 5% by weight, typically less than 3% by weight, more typically less than 1 % by weight of non-specified components.

The term “consisting of” or “consists of” means including the components specified and excluding the addition of other components.

Whenever appropriate, depending upon the context, the use of the term “comprises” or “comprising” may also be taken to encompass or include the meaning “consists essentially of” or “consisting essentially of”, and may also be taken to include the meaning “consists of” or “consisting of”. The term “comprising” may in embodiments be substituted by the term “consisting of” or “consisting essentially of”.

The optional features set out herein may be used either individually or in combination with each other where appropriate and particularly in the combinations as set out in the accompanying claims. The optional features for each aspect or exemplary embodiment of the invention as set out herein are also to be read as applicable to any other aspect or exemplary embodiments of the invention, where appropriate. In other words, the skilled person reading this specification should consider the optional features for each exemplary embodiment of the invention as interchangeable and combinable between different exemplary embodiments.

In this specification, the term “anti-microbial” refers to a compound or a composition that may kill and/or inhibit and/or stop the growth of any one or more types of microorganisms, including, viruses, prions, protozoa, amoeba, bacteria, fungi and yeasts, or any one or more of specific species of microorganism.

The term “biofilm” refers to both a monocultured and a polymicrobial community of microorganisms enclosed in an extracellular polymeric matrix (composed of EPS), and attached to a biotic or an abiotic surface.

The term “biofilm formation” refers to the attachment of microorganisms to surfaces and the subsequent development of multiple layers of cells within an EPS matrix.

The term “anti-biofilm” refers to the inhibition of microbial biofilm formation and/or disruption and/or dispersal of biofilms and/or detachment and/or dispersion and/or breakdown of EPS of a biofilm.

The term “anti-inflammatory” refers to the property of a substance or treatment that reduces inflammation or swelling, typically in a wound.

The term “infection” refers to the invasion and multiplication of microorganisms such as bacteria, viruses, fungi, yeasts and parasites that are not normally present within the body. An infection may cause no symptoms and be subclinical, or it may cause symptoms and be clinically apparent. An infection may remain localized, or it may spread through the blood or lymphatic vessels to become systemic (body wide). Microorganisms that live naturally in the body are not considered infections.

The term “wound” includes a type of injury in which skin is torn, cut or punctured (an open wound), or where blunt force trauma causes a contusion (a closed wound).

The term “acute wound” refers to those wounds which are new and in the first phase of healing. Acute wounds are characterized by skin layers that have been punctured or broken by an external force or object. Any acute wound can progress to a chronic wound if it does not heal within the expected time frame or as a result of a poor supply of blood, oxygen, nutrients or through poor hygiene. Acute wounds should be properly treated to avoid infection and/or inflammation. Acute wounds are categorized based on causes such as lacerations, abrasions, punctures, incisions, gunshots, burns and according to their size and depth (superficial or deep).

The term “chronic wound” refers to a wound that will not repair itself over time. Chronic wounds are often thought to be “stuck” in one of the phases of wound healing, and are most often seen in the older adult population. Typically, if a wound is not healing as expected within 2-3 months, it is considered chronic. Chronic wounds include pressure ulcers (e.g. bed sores), arterial and venous leg ulcers, and diabetic ulcers.

### DESCRIPTIONS OF THE FIGURES

Embodiments of the present invention will now be described, by way of example only, with reference to the accompanying Figures in which:

FIG. 1 is a powder X-Ray diffractogram of Ag₃CuDTPA.

FIG. 2 is a power X-Ray diffractogram of copper sulphate.

FIG. 3 is a powder X-Ray diffractogram of silver nitrate.

FIG. 4 is a powder X-Ray diffractogram of DPTA.

### EXAMPLES

The invention, and the surprising benefits afforded over the prior art, will be illustrated with reference to the following non-limiting examples.

As mentioned above, it has been unexpectedly observed that the efficacy of the metal aminopolycarboxyl compounds of the present invention are significantly greater than the EDTA based compounds described in WO2017/191453. This is evidenced by the minimum inhibitory concentration (MIC) data provided below.

### Preparation of Compounds of the Present Invention

Compounds of the present invention may be suitably synthesised according to the following exemplary synthesis methods.

### Method 1 - Preparation of Ag₃Cu(DTPA) (Analogous to the Methods Disclosed in WO2017/191453A1)

A stock solution of DTPA sodium salt was made by dissolving 1.68 g DTPA in 18.12 g demineralised water plus 1.2 g of 40% aqueous NaOH solution. 3.6 g of the DTPA solution was added to a solution of 0.6 g silver nitrate (AgNO₃) made up to 3.5 g with demineralised water and the mixture shaken for 30 seconds. The resulting milky white liquid was then centrifuged at 2500 rpm for 3 minutes. The supernatant liquid containing sodium nitrate by-product was then removed using a pipette leaving a solid containing the silver/DPTA complex. The silver/DPTA solid was then dissolved in a solution of 0.47 g copper sulphate pentahydrate (CuSO₄·5H₂O) made up to 9.34 g with demineralised water to form the final compound Ag₃Cu(DPTA).

### Method 2 - Preparation of Ag₃Cu(DTPA) Without the Step of Centrifugation Separation

A stock solution of DTPA sodium salt was made by dissolving 1.68 g DTPA in 18.12 g demineralised water plus 1.2 g of 40% aqueous NaOH solution. 3.6 g of the DTPA solution was added to a solution of 0.6 g silver nitrate (AgNO₃) made up to 3.5 g with demineralised water and the mixture shaken for 30 seconds. The resulting solution was then directly combined with a solution of 0.47 g copper sulphate pentahydrate (CuSO₄·5H₂O) made up to 9.34 g with demineralised water to form the final compound Ag₃Cu(DPTA).

### Method 3 - Preparation of Ag₃Cu(DTPA) Where Copper Sulphate (CuSO₄·5H₂O) is Introduced Before Silver Nitrate (AgNO₃)

0.84 g DTPA was dissolved in 2.66 g NaOH solution containing demineralised water and 0.58 g 40% of NaOH to produce a DTPA sodium salt solution. 1.8 g silver nitrate (AgNO₃) was dissolved in 1.7 g demineralised water to produce a silver nitrate solution. 1.41 g copper sulphate pentahydrate (CuSO₄·5H₂O) was dissolved in 7.93 g demineralised water to produce a copper sulphate solution. Copper sulphate solution was added to the DTPA solution with vigorous stirring. A dark blue clear solution was formed. Silver nitrate solution was added to the clear blue solution to form the final compound Ag₃Cu(DTPA). After stirring the mixture remained as a dark blue clear solution.

It was found that when performing a method using identical starting materials and reagents to that of method 3 above, but instead introducing the silver nitrate solution to the DTPA sodium salt solution before then adding the copper sulphate solution, a milky white dispersion was formed containing a solid DPTA/silver intermediate species. Method 3 involves the addition of the copper sulphate solution to the DTPA sodium salt solution first forms a dark blue clear solution containing a DPTA/copper intermediate species. This sequence of method steps is advantageous since all materials remain in solution during the entire method, which as a result, improves reaction times and yields. In addition, this is advantageous when working on larger scales since there is no need for an intermediate dissolution step to aid the solubility of the DPTA silver intermediate species. Higher concentrations of final compound solutions are achievable when using method 3 described above.

A further variant of the preparation process may also involve pre-mixing the two or more metal salt solutions (e.g. silver nitrate solution and copper nitrate solution) be adding the mixed metal salt solution to the DTPA sodium salt solution. The skilled person understand that this is only desirable where the two or more metal salts solutions are miscible. One particular example of this has been observed using a combined mixture of copper nitrate solution and silver nitrate solution which forms a miscible mixture of silver and copper. This can then be used with a DTPA sodium salt solution to successfully form a silver/copper DTPA compound according to the present invention.

### Concentration Studies

### Ag₃CuDPTA

1.53 g DTPA was dissolved in 4.37 g demineralised water plus 2.10 g 40% NaOH to produce a DTPA sodium salt solution. 3.29 g silver nitrate was dissolved in 4.71 g demineralised water to produce a silver nitrate solution. 2.58 g copper sulphate pentahydrate was dissolved in 16.09 g demineralised water to produce a copper sulphate solution. Silver nitrate solution was added to the DPTA solution with vigorous stirring. A milky white dispersion was formed. Copper sulphate solution was added to the white dispersion. With vigorous stirring the white precipitate dissolved to give a clear, blue liquid, signifying the formation of the silver/copper DTPA mixed metal complex (MMC). The total soluble solids content of the solution was measured and found to be 22.5% w/w. The theoretical concentration of silver/copper DTPA MMC is 9.2% (0.15 mol/Kg). Comparative concentration studies performed on Ag/Cu EDTA MMC examples provided theoretical concentration values of 0.07-0.09 mol/Kg.

### Ag₃ZnDPTA

1.11 g DTPA was dissolved in 4.59 g demineralised water plus 1.3 g 40% NaOH to produce a DTPA sodium salt solution. 2.39 g silver nitrate was dissolved in 4.61 g demineralised water to produce a silver nitrate solution. 2.08 g zinc sulphate monohydrate was dissolved in 15.92 g demineralised water to produce a zinc sulphate solution. Silver nitrate solution was added to the DTPA solution with vigorous stirring. A milky white dispersion was formed. Zinc sulphate solution was added to the white dispersion. With vigorous stirring the white precipitate dissolved to give a clear, light brown liquid, signifying the formation of silver/zinc-DTPA mixed metal complex. The total soluble solids content of the solution was measured and found to be 24.7% w/w. The theoretical concentration of silver/copper DTPA MMC is 6.8% (0.11 mol/Kg). Comparative concentration studies performed on Ag/Zn EDTA MMC examples provided theoretical concentration values of 0.04-0.06 mol/Kg

These concentration studies illustrate that the DPTA complexes of the present invention is capable of being loaded into an aqueous solution at a high concentration as compared to their EDTA equivalents.

### Preparation of Ag₂Al(DTPA)

0.29 g DTPA was dissolved in 2.93 g demineralised water plus 0.28 g of 40% NaOH to produce a DTPA sodium salt solution. 0.24 g silver nitrate (AgNO₃) was dissolved in 3.26 g demineralised water to produce a silver nitrate solution. 0.66 g aluminium sulphate hexadecahydrate (Al₂H₃₂O₂₈S₃) was dissolved in 8.34 g demineralised water to produce an aluminium sulphate solution. Silver nitrate solution was added to the DTPA solution with vigorous stirring. A milky white dispersion was formed. Aluminium sulphate solution was added to the white dispersion. With vigorous stirring the white precipitate dissolved to give a clear, colourless liquid, signifying the formation of Ag₂Al(DTPA) mixed metal complex.

The total soluble solids content of the solution was measured and found to be 7.2% w/w. The theoretical concentration of Ag₂AlDTPA is 2.9%.

### Preparation of Ag₂Bi(DPTA)

A DTPA sodium salt solution was formed by dissolving 1 g DTPA in 10.4 g demineralised water plus 0.6 g of 40% NaOH solution. 0.15 g bismuth nitrate (Bi(NO₃)₃)0.5H₂O was added to the DTPA solution and stirred for 1 hour until the bismuth nitrate dissolved to form a bismuth/OPTA solution. Silver nitrate solution was formed by dissolving 0.24 g silver nitrate (AgNO₃) in 3.26 g demineralised water. 0.62 g of the silver nitrate solution was added dropwise to 3 g of the bismuth/DTPA solution whilst stirring. At this point a clear and colourless solution was formed. A theoretical total solids concentration is 9.7%.The theoretical concentration of Ag₂BiDTPA is 0.02 mol/Kg.

### General Outline of MIC Measurement Method

MIC measurements were performed using an adapted method described in the Clinical and Laboratory Standards Institute (CLSI) guidelines M07-A11. Compounds were serial diluted two-fold in Mueller Hinton Broth (MHB) in 96 well plates. A positive growth control and a negative growth control were also included in the well plates.

A colony suspension of S. aureus (ATCC 29213), P. aeruginosa (ATCC 15442) and C. albican was prepared by taking several colonies from a fresh agar plate and suspending them in MHB. The suspension was adjusted to 0.5 McFarland (~1 × 10⁸ CFU/mL) and diluted 1:100. The inoculum was then added to wells at a final concentration of ~5 × 10⁶ CFU/mL. Plates were incubated overnight at 37° C. The following day, MICs were determined as the lowest concentration that no growth could be visually observed.

### Anti-Biofilm Ability - MIC Measurements

### MIC Measurements for Uncomplexed DPTA and EDTA Compounds

An 80 mg/ml solution of T-EDTA was prepared by dissolving T-EDTA (99% pure, Acros Organics) in sterile distilled water. The pH of this solution was measured at 10.5, using a pH electrode. A 16 mg/ml solution of DTPA was prepared by dissolving DTPA (98%+ Acros Organics) in distilled water, and adjusting pH to 10.5 using the addition of sodium hydroxide.

Table 1 shows the MIC results measured for both T-EDTA and DTPA compounds (which are not complexed to metal ions) as well as the metal aminopolycarboxyl compounds Ag₂Cu(EDTA), Ag₃Cu(DPTA), Ag₂Al(DTPA) and Ag₂Bi(DTPA). It can be seen that only modest increases in potency against P. aeruginosa and S. aureus are observed (2.5 and 1.25 fold respectively) for the uncomplexed DTPA as compared to the uncomplexed T-EDTA. In contrast, the results show that when comparing MIC potency measurements for Ag₂Cu(EDTA) and Ag₃Cu(DPTA) there is a significant increase in potency for the DPTA compound. In particular, there is an approximately 16 fold increase against S. aureus and an approximately 4 fold increase against P. aeruginosa when comparing the MIC values for Ag₃Cu(DPTA) versus the prior art compound Ag₂Cu(EDTA). This would not have been expected based on the MIC results for both T-EDTA and DTPA compounds.

TABLE 1

| | P. aeruginosa (ATCC 15442) | S. aureus (ATCC 29213) | C. albican (ATCC 10231) |
|---|---|---|---|
| | MIC (µg/ml) | MIC (µg/ml) | MIC (µg/ml) |
| T-EDTA | 5000 | 1250 | - |
| DTPA | 2000 | 1000 | - |
| Ag₂Cu(EDTA) | 6 | 98 | - |
| Ag₃Cu(DPTA) | 1.6 | 6 | - |
| Ag₂Al(DTPA) | 450 | 370 | 530 |
| Ag₂Bi(DTPA) | 94 | 190 | 11 |

### Minimum Inhibitory Concentration (MIC) Assay for AgZnDTPA and AgCuDTPA Complexes With Non-Metal Additives

The MIC values for AgZnDTPA and AgCuDTPA (with and without additives) were determined against Staphylococcus aureus ATCC 29213 and Pseudomonas aeruginosa ATCC 15442. The DTPA complexes compositions with and without additives were serial diluted two fold in Mueller Hinton Broth (MHB) in 96 well plates as mentioned above. The plates were then inoculated with the bacterial strains as described above and the following day, MICs were determined as the lowest concentration that no growth could be visually observed.

### Results

AgZnDTPA and AgCuDTPA showed potent antimicrobial activity against both bacterial strains with MICs 0.016 against S. aureus and 0.0009 against P. aeruginosa. Following combination of additives such as Tris and Urea an 80 fold and 9 fold increase in MIC of both DTPA complexes were found against S. aureus and P. aeruginosa, respectively. Additionally, combination of the DTPA complexes with antimicrobials at subtherapeutic concentrations (0.25x MIC) was found to increase the potency of the complexes, with iodine increasing the MIC by ≥160 fold against both bacterial strains and hypochlorous acid (Suprox) increasing the potency 9 and 16 fold against P. aeruginosa and S. aureus, respectively.

TABLE 2

| Bacterial Strain | Complex | Additive | MIC | Fold Change |
|---|---|---|---|---|
| S. aureus ATCC 29213 | AgZnDTPA | No additive | 0.016 | - |
| | | 1.5% Tris | 0.0002 | 80 |
| | | 25% Suprox | 0.001 | 16 |
| | | 0.04% Iodine | ≤0.0001 | ≥160 |
| | AgCuDTPA | No additive | 0.016 | - |
| | | 1.5% Tris | 0.0002 | 80 |
| | | 25% Suprox | 0.001 | 16 |
| | | 0.04% Iodine | ≤0.0001 | ≥160 |
| P. aeruginosa ATCC 15442 | AgZnDTPA | - | 0.0009 | - |
| | | 1.5% Urea | 0.0001 (9 fold) | 9 |
| | | 50% Suprox | 0.0001 (9 fold) | 9 |
| | | 0.04% Iodine | ≤0.00002 | ≥45 fold |
| | AgCuDTPA | - | 0.0009 | - |
| | | 1.5% Urea | 0.0001 (9 fold) | 9 |
| | | 50% Suprox | 0.0001 (9 fold) | 9 |
| | | 0.04% Iodine | ≤0.00002 | ≥45 fold |

### Log Reduction Assay for AgZnDTPA and AgCuDTPA Complexes With Non-Metal Additives

The speed of kill of two DTPA metal complexes, AgZnDTPA and AgCuDTPA was evaluated against S. aureus ATCC 29213 and P. aeruginosa ATCC 15442. The DTPA complexes were diluted to 8x and 16x MIC in MHB and inoculated with the strains at a final bacterial cell density of 1 × 106 CFU/mL. Tubes were incubated at 37° C. and 125 rpm for 24 hours and samples were collected at 3 hours and 24 hours. Collected samples were added to Dey-Engley neutralising broth at a 1:10 ratio, vortexed and serial diluted 1:10 in PBS before spot plating the dilutions onto trypticase soy agar (TSA). Plates were incubated overnight at 37° C. and the following day bacterial colonies were enumerated. The log reduction was calculated as the difference in bacterial cell density in treated samples compared to untreated growth controls.

### Results

At 16x MIC (0.256%) both DTPA complexes showed complete eradication of S. aureus by 24 hours, with a 9.1 log reduction in bacterial cell density. At 16x MIC (0.0072%) both DTPA complexes showed complete eradication of P. aeruginosa by 3 hours, with a 7 log reduction in bacterial cell density. Following addition of 1.5% Tris an enhanced speed of kill was found with AgZnDTPA at 8x MIC, with complete eradication of S. aureus by 3 hours (7.1 log reduction).

### Conclusion

The mixed metal complexes AgZnDTPA and AgCuDTPA both showed potent antimicrobial activity against representative Gram-positive and Gram-negative strains S. aureus and P. aeruginosa. Enhanced antimicrobial activity was found when additives or antimicrobials at subtherapeutic concentrations such as Tris, Urea, Hypochlorous Acid and Iodine were included.

Additionally, the DTPA complexes demonstrated rapid speed of kill, with eradication of P. aeruginosa by 3 hours and S. aureus by 24 hours. The speed of kill of S. aureus was increased following inclusion of an additive 1.5% Tris, with eradication found by 3 hours.

### Structural Characterisation of the Compounds of the Present Invention

Compounds of the present invention have been characterised using X-Ray Powder Diffraction (XRD) techniques known to the person skilled in the art.

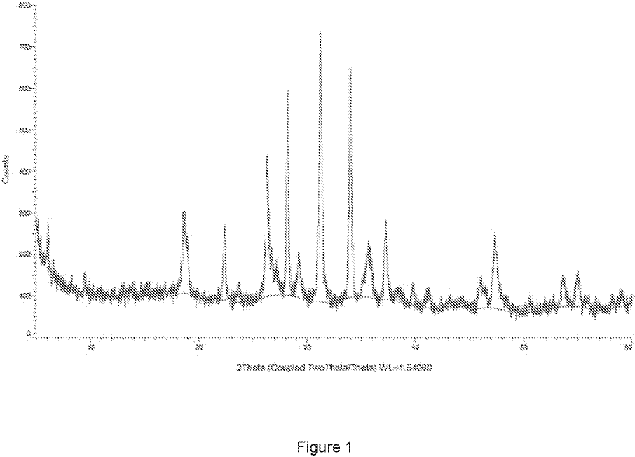

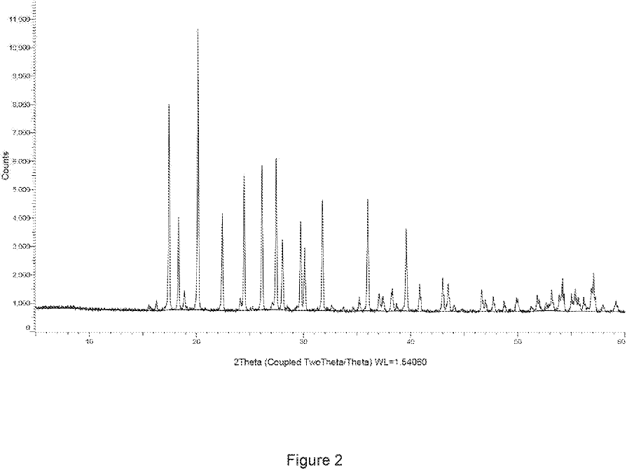

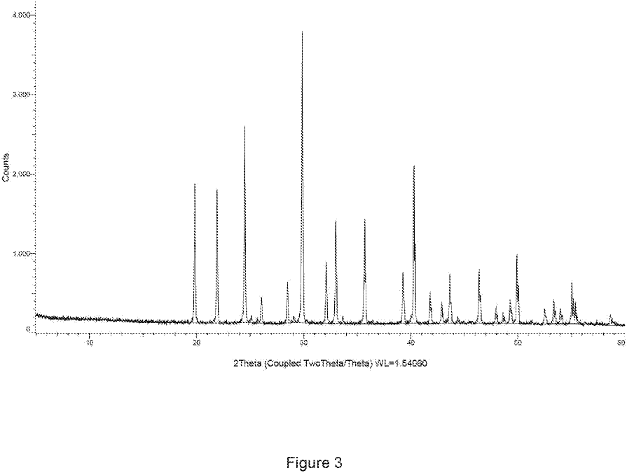

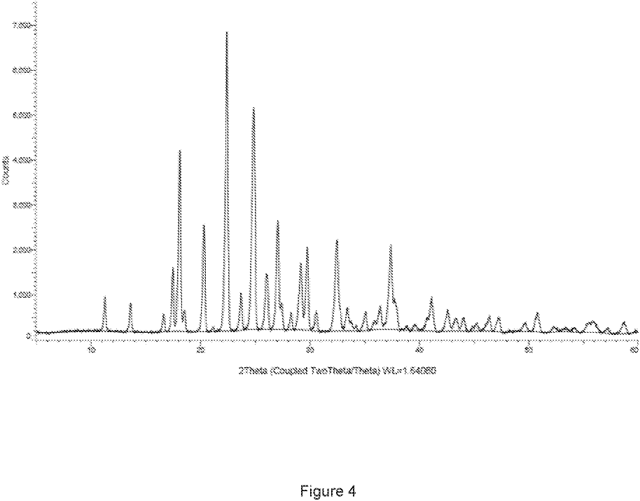

FIG. 1 illustrates an X-Ray powder diffractogram of Ag₃CuDTPA prepared according to the preparation method 3 described above with the additional steps of drying the product in a recirculating oven at 60° C. to constant weight, then turning the raw solid into a powder for diffraction analysis. FIGS. 2 to 4 show X-Ray diffractograms measured for the starting materials copper sulphate, silver nitrate and DPTA. When comparing the diffractogram of Ag₃CuDTPA with the raw materials DTPA, copper sulphate, silver nitrate it is evident that the diffractogram for the Ag₃CuDTPA complex contains peaks at different positions to the raw materials and that the peaks present in the starting material diffractograms are generally absent. For example, the major reflections of DTPA at 20.5, 24.9 and 32.5 2-theta degrees are substantially absent from the Ag₃CuDTPA diffractogram and the major reflections of silver nitrate at 29.8 two-theta degrees and copper sulphate at 20.2 two-theta degrees are completely absent. The X-Ray powder diffractogram data confirms the formation of the Ag₃CuDTPA complex according to the present invention.

1. A compound of formula Mₙ(P), wherein
n is an integer from 2 to 6;
each M is independently a metal ion;
Mn comprises at least two different metal ions selected from Ag, Al, Au, Ba, Bi, Tl, Ce, Co, Cu, Fe, Ga, Ge, Ir, Mo, Rh, Ru, Sb, Se, Sn, Sr, Ti and Zn ions; and
P is an aminopolycarboxyl component comprising an optionally substituted alkylene amino backbone containing from 3 to 5 nitrogen atoms in the backbone and 5 or 6 carboxyl groups appended to the backbone, wherein the aminopolycarboxyl component contains from 10 to 20 atoms in the longest linear chain.

2. The compound according to claim 1, wherein P is a compound according to formula (I): 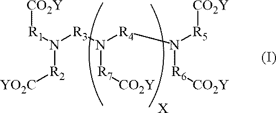
wherein:
X is 1 or 2
each Y group is independently H or a negative charge wherein at least two Y groups are negative charges; and
R₁, R₂, R₃, R₄, R₅, R₆ and R₇ are each independently optionally substituted C₁₋₃alkylene.

3. The compound according to claim 2, wherein R₃ and R₄ are each optionally substituted ethylene and/or wherein R₁, R₂, R₅, R₆ and R₇ are each optionally substituted methylene or ethylene.

4. The compound according to claim 3, wherein R₃ and R₄ are each ethylene and R₁, R₂, R₅, R₆ and R₇ are each methylene.

5. The compound according to claim 1, wherein Mₙ comprises at least one Ag ion and at least one Cu ion or wherein Mn comprises at least one Ag ion and at least one Zn ion.

6. The compound according to claim 1, wherein the compound is selected from AgCu(DPTA), AgZn(DPTA), AgAl(DPTA), AgBi(DPTA), AgMo(DPTA), AgSr(DPTA), Ag₂Cu(DPTA), Ag₂Zn(DPTA), Ag₂Al(DPTA), Ag₂Bi(DPTA), Ag₃Cu(DPTA), Ag₃Zn(DPTA), AgCu(TTHA), AgZn(TTHA), AgAl(TTHA), AgBi(TTHA), AgMo(TTHA), AgSr(TTHA) Ag₂Cu(TTHA), Ag2Zn(TTHA), Ag₂Al(TTHA), Ag₂Bi(TTHA), Ag₃Cu(TTHA), Ag₃Zn(TTHA), Ag₃Al(TTHA) Ag₃Bi(TTHA), Ag₄Cu(TTHA) and Ag₄Zn(TTHA).

7. A composition comprising the compound of claim 1 and one or more pharmaceutically acceptable excipients.

8. The composition according to claim 7, wherein the composition is a hydrogel or an aqueous solution, a lotion, an ointment, a cream, a balm, a gel, a paste or a solid.

9. The composition according to claim 7, wherein the composition comprises fibres which are in contact with the one or more compounds, wherein the one or more compounds are provided on the surface of the fibres, and-/-or wherein the one or more compounds are incorporated within the fibres.

10. The composition according to claim 7, wherein the composition further comprises a non-metal ion anti-microbial agent, surfactant, and/or further metal ion chelator.

11. A method of forming a compound of formula Mₙ(P), wherein
n is an integer from 2 to 6;
each M is independently a metal ion;
Mn comprises at least two different metal ions selected from Ag, Al, Au, Ba, Bi, Tl, Ce, Co, Cu, Fe, Ga, Ge, Ir, Mo, Rh, Ru, Sb, Se, Sn, Sr, Ti and Zn ions; and
P is an aminopolycarboxyl component comprising an optionally substituted alkylene amino backbone containing from 3 to 5 nitrogen atoms in the backbone and 5 or 6 carboxyl groups appended to the backbone, wherein the aminopolycarboxyl component contains from 10 to 20 atoms in the longest linear chain,
the method comprising:
providing a solution comprising P;
contacting the solution comprising P with a first metal ion source comprising a first metal ion M and a second metal ion source comprising a second metal ion M to form the compound of formula Mₙ(P);
wherein the first metal ion M and second metal ion M are independently selected from Ag, Al, Au, Ba, Bi, Tl, Ce, Co, Cu, Fe, Ga, Ge, Ir, Mo, Rh, Ru, Sb, Se, Sn, Sr, Ti and Zn ions, and the first metal ion M is different to the second metal ion M.

12. The method according to claim 11, wherein:
the first metal ion source is an Ag metal ion source and the second metal ion source is a Cu metal ion source; or
the first metal ion source is a Cu metal ion source and the second metal ion source is a Ag metal ion source; or
the first metal ion source is an Ag metal ion source and the second metal ion source is a Zn metal ion source;
or wherein the first metal ion source is a Zn metal ion source and the second metal ion source is a Ag metal ion source.

13. A method of preparing an anti-microbial composition comprising at least two different metal ions selected from Ag, Al, Au, Ba, Bi, Tl, Ce, Co, Cu, Fe, Ga, Ge, Ir, Mo, Rh, Ru, Sb, Se, Sn, Sr, Ti and Zn ions and a component P, wherein P is an aminopolycarboxyl component comprising an optionally substituted alkylene amino backbone containing from 3 to 5 nitrogen atoms in the backbone and 5 or 6 carboxyl groups appended to the backbone, wherein the aminopolycarboxyl component contains from 10 to 20 atoms in the longest linear chain, the method comprising:
providing a solution comprising component P;
contacting the solution comprising component P with a first metal ion source comprising a first metal ion M selected from Ag, Al, Au, Ba, Bi, Tl, Ce, Co, Cu, Fe, Ga, Ge, Ir, Mo, Rh, Ru, Sb, Se, Sn, Sr, Ti and Zn ions; and a second metal ion source comprising a second metal ion M that is different to the first metal ion M, the second metal ion M selected from Ag, Al, Au, Ba, Bi, Tl, Ce, Co, Cu, Fe, Ga, Ge, Ir, Mo, Rh, Ru, Sb, Se, Sn, Sr, Ti and Zn ions, to form the anti-microbial composition;
wherein the molar ratio of the component P, first metal ion source and second metal ion source (P : first metal ion : second metal ion) is from about 1 : 2 : 2 to about 1 : 10 : 10 or from about 1 : 0.1 : 0.1 to about 1 : 10 : 10.

14. An anti-microbial composition obtained by the method according to claim 13.

15. A wound dressing or medical device comprising the compound according to claim 1.

16. A method for sanitising or substantially removing a biofilm from a substrate, the method comprising contacting the substrate with the compound according to claim 1, wherein the method excludes using the compound or composition in a method for treatment of the human or animal body by surgery or therapy.

17. (canceled)

18. The method of claim 16, wherein the substrate is a wound on a human or animal body.

19. A method for treating infections of cuts, bruises, surgical sites, lacerations, abrasions, punctures, incisions, gunshots, bums, pyoderma, atopic dermatitis, eczema, pressure ulcers, venous and artery leg ulcers, diabetic foot ulcers, cystic fibrosis (CF)-associated infections, mastitis, otitis, community or hospital acquired infections, or food-borne diseases in a subject in need thereof the method comprising treating the subject with a pharmaceutically effective amount of the compound according to claim 1.
